(19) United States
(12) Patent Application Publication
(10) Pub. No.: US 2010/0152622 A1
(43) Pub. Date: Jun. 17, 2010

(54) MOTOR PHENOMENON MEASUREMENT

(76) Inventor: Johannes Leonardus Hermanus Maria Teulings, Tempe, AZ (US)

Correspondence Address: THE LUTHER LAW FIRM, PLC, 10575 N 114TH STREET, SUITE 103, SCOTTSDALE, AZ, 85259, US

(21) Appl. No.: 12/600,464

(22) PCT Filed: May 16, 2008
(86) PCT No.: PCT/US08/06292

§ 371 Date: Nov. 16, 2009

Related U.S. Application Data
(60) Provisional application No. 60/938,409, filed on May 16, 2007.

(51) Int. Cl. A61B 5/11; G06Q 50/00

(52) U.S. Cl. 600/595; 705/2

(57) ABSTRACT

Motor phenomena are measured by using a pen-and-tablet digitizer and a personal computer to instruct, prompt and make a data record of a human patient's performance of a handwriting task, analyze the data record, and report features of the patient's performance. Examples of reported features are measures of dysfluency and measures of velocity scaling. Reported features are correlated with information about the patient's state of health (especially schizophrenia), medication (especially antipsychotic and neuroleptic) as a superior alternative to scales based on clinical observation for detecting motor effects of a medication, comparing side effects of medications, adjusting dosages, and observing the course of motor disturbances such as Parkinsonism and tardive dyskinesia.

### TECHNICAL FIELD

The present invention relates generally to measuring an effect of a drug on a human being, more particularly to monitoring side effects of antipsychotic and neuroleptic drugs in schizophrenic patients, and especially to analyzing a medicated patient's performance of a motor task for extrapyramidal motor signs characteristic of drug toxicity in order to alter a course of treatment and to abate drug-induced motor disturbance.

### BACKGROUND ART

The use of handwriting to assess extrapyramidal sideffects (EPS) was first reported by Haase (1961; and later in 1978) and was adopted in more recent psychopharmacological studies (Gerken et al., 1991; Gallucci et al., 1997; Kuenst et al., 1999). Gerken et al. (1991) expressed movement size as the area encompassed by handwriting in schizophrenic patients. Handwriting area decreased by an average of 13% following antipsychotic treatment in a majority of patients studied. The authors concluded that handwriting parameters could be better suited for evaluating neurological side effects of neuroleptic medication to predict treatment response than the conventional observer-based severity rating scales. Gallucci et al. (1997) observed an association between handwriting duration consistency and medication type in their schizophrenia patients. Kuenstler et al. (1999) used positron emission tomography (PET) in schizophrenic patients before and after treatment with drugs (haloperidol, clozapine, or risperidone). They found a significant relationship between reduction in handwriting size (expressed by area) and dopamine receptor occupancy suggesting that handwriting size decreased in proportion to the number of dopamine receptors that were blocked by the actions of these drugs. Drug-induced motor side effects are thought to stem from the dopamine receptor blocking properties of antipsychotic medications. Collectively, these studies suggest that handwriting parameters might be better suited for evaluating neurological side effects of neuroleptic medication than predicting treatment response using observer-based severity rating scales.

U.S. Pat. No. 6,632,174 to Breznitz, (2003) describes testing and training cognitive ability, including the steps of testing a preliminary cognitive level of a user, receiving representative results, and using those results to break the cognitive level into separate discrete cognitive skills, and creating tasks related to the separate discrete cognitive skills. The one or more tasks may then be presented to the user and so that a current cognitive level of the user is re-tested, and so on.

U.S. Pat. No. 6,546,134 to Shrairman and Landau (2003) describes biometric assessment of human fine motor control through analysis of forces and accelerations during cursive writing. Criteria of stability, smoothness and synchronization of the writer's motion are returned as measures of neurological function. The method processes behavioral random signals with application of correlation function analysis to handwriting dynamic signals, returning numerical criteria scores and graphical displays showing stability of handwriting strokes and characteristics of phase distortions in reproducing cursive samples. The method can be applied to monitoring patients with neurological disorders, monitoring people with alcohol/drug abuse problems, detection of fine motor control deterioration as a result of exposure to toxic materials, and testing the efficacy of countermeasures to improve impaired fine motor control functions.

U.S. Pat. No. 6,454,706 to Pullman (2002) describes computerized clinical assessment of motor function by correlating geometric indices, computed from digital information obtained from a geometric shape drawn by a subject to be evaluated, with a rating score derived using a “standard of reference” generated by one or more clinical expert. Interpretation is thereby rendered more objective and consistent.

U.S. Pat. No. 5,772,611 to Hocherman (1998) describes detection and quantification of Parkinson's disease. They used a stylus moving together with a handle and adapted to produce signals sensible by the digitizer tablet, and means to produce on the monitor screen a model path of a predetermined shape and size, as well as a target to be tracked by the subject, the target moving at a predetermined speed along the model path. Detection and quantification of Parkinson's disease is also described and claimed.

Verifax, Colorado, USA Neuroskill™ software for biometric measurement, security purposes, and Parkinson medication effects is focused on the measurement of Parkinsonian movement disorders and will remain so. Verifax is operated by Ruth Shrairman and Alexander Landau, grantees of aforementioned U.S. Pat. No. 6,546,134. Their applications are substance abuse screening and detection, monitoring for toxic inhalants and environmental distress, and accurate signature identification for security/privacy protection and forgery detection. Target markets could include neuromuscular disease centers, drug and alcohol abuse clinics, occupational health centers and the security industry. The Verifax method uses a new approach for Correlation Function Analysis applied to behavioral signals, such as handwriting dynamics. KikoSoft (kikosoft.com), The Netherlands, Oasis™ software for Microsoft DOS Windows 95/98 or special hardware. Oasis has been designed by Peter de Jong (See De Jong et al., 1996): Oasis is a universal, programmable system with its macro syntax allowing the user to implement a large variety of interactive tests. Orgabat™ is that company's test battery for psychopharmacological research.

Science And Motion (scienceandmotion.com), Germany CS Win™ (See Mai and Marquardt, 1999), marketed in Germany since about 1990 and used in many German hospitals. It was used for treating 500 writer's cramp patients.

MedDraw (meddraw.org) is a European-funded project in UK and France using drawing movement tests to detect spatial neglect in the visual field and organization of movement disorder. This project will remain focused on drawing-based diagnosis of these disorders. It started in 2003 and involves 10 researchers including Guest, Fairhurst, and Heutte.

The Actometry and Barnes Akathisia Rating Scale in neuroleptic-induced akathisia (Janno, Holi, Tuisku, Wahlbeck, 2005a; 2005b) (Barnes Akathisia Rating Scale, “BARS”) and standardized lower limb actometry were used to quantify neuroleptic-induced akathisia (NIA) in 99 schizophrenia patients. Both instruments discriminated well between NIA and non-NIA patients and they correlated weakly but significantly. The visual observation scale BARS was superior to actometry in screening DSM-IV diagnosed NIA patients, though. The results of this methodological study provide BARS with objective validation through movement measuring. Actigraphy has been in use for several years, mostly for sleep studies (wrist worn device that measures amount of activity at night, or reduced activity during the day (napping)). Many published reports of activity monitoring in adults, children, nursing home, exist.

### SUMMARY OF INVENTION

### Technical Problem

Many nervous and mental disorders exist in humans that effect their movements. Additionally, medicines used to treat these conditions can adversely effect movements. For demonstration purposes we focus on schizophrenia. World-wide, 1% of the population is affected by schizophrenia. Standard treatment for schizophrenia includes the use of powerful antipsychotic medications which are known to cause irreversible movement disorders in 15%-20% of individuals taking these medicines.

Managing these adverse conditions requires careful assessment. Conventional clinical observation tests are visual observations of the patient's movements on ordinal scales. The inventors concluded that these conventional ratings fail to show differences between different types of medications or between different dosages of a given type of medication. Moreover, human movements in the natural setting are difficult to express by conventional observation tests.

Neuroleptic medications have been the mainstay for treating psychotic illness for over 50 years. While neuroleptics improve the lives of schizophrenic patients, the occurrence of extrapyramidal motor signs (EPS) with increasing dosage will limit some patients in reaching dosages that are effective to treat the disease. Even after the emergence of the newer second generation of antipsychotics, EPS continue to cause concern, particularly in vulnerable populations, such as the elderly (Caligiuri et al., 2000).

The mechanism of action by which antipsychotic medications lead to a reduction in psychosis generally involve interrupting the transmission of dopamine throughout the frontal-striatal pathways in the brain. Conventional antipsychotics block the same dopamine receptors that are important in motor function and in doing so they produce motor disturbances such as parkinsonism.

Chronic dopamine receptor blockade can lead to permanent damage to these pathways resulting in tardive dyskinesia (TD), a disfiguring condition characterized by uncontrolled orofacial and hand movements. Epidemiologic studies indicate that nearly 60% of patients treated with conventional neuroleptic drugs develop EPS such as parkinsonism, while 20-25% of the patients develop irreversible tardive dyskinesia (TD) (Caligiuri et al., 2000). These serious conditions might have been prevented if EPS could have been detected early in the management of the patient's pharmacotherapy. Monitoring EPS could improve treatment outcome, compliance and reduce re-hospitalization.

Effective management of EPS begins with early detection, and eventually, prevention. Early detection requires sensitive and reliable measurement, while prevention requires knowledge of risk factors and experience. The ability to monitor the severity and course of EPS is critical to the understanding and management of drug-induced side effects.

The lack of reliable and precise EPS assessment methods forms a bottleneck in delineating the syndrome, determine its prevalence, and evaluate the efficacy of treatments. These problems have prompted researchers to search for more reliable, quantitative, instrumental methods for assessing EPS.

### Solution to the Problem

The present invention uses exact measurements of handwriting movements (i.e., the movement of the pen tip on and above the writing surface as a function of time) and other fine motor skills to assess EPS. Handwriting is recorded using such items as low cost, off-the-shelf graphic digitizing tablets. A PC-based software system analyzes pen movement data recorded by the digitizer. Several specific pen movement tasks and several specific analysis methods yield objective measures that significantly change with medication type and with dosage of a given type of medication. The movement tasks are easy-to-perform, repetitive circle and loop patterns. These objective measures are based on well-defined algorithms. The algorithms are analytic and not adaptive so they remain the same over time. The various measures express different aspects of the movement: dysfluency of movement, velocity scaling (i.e., to what extent a person increases velocity when required to increase size), and bradykinesia (i.e., movement slowness). Absolutely novel in the present application is the use of movement dysfluency and other movement measures to quantify a patient's response to certain types of medication.

### ADVANTAGEOUS EFFECTS OF INVENTION

In contrast to the '134 Shrairman patent, the present invention uses simple, well established human movement features. Shrairman uses a particular correlation function analysis. Another difference is that the present invention shows significant effects due to medication type and dosage, proving its efficacy.

In contrast to the '706 Pullman patent, the present invention provides a means for evaluating persons early in the course of disease and for screening patients for motor dysfunction or, in the case of children, disorders of motor development.

Also in contrast to the '706 Pullman patent, the present invention uses voluntary handwriting movements. Pullman uses geometrical shapes.

In contrast to the '611 Hocherman patent, the present invention uses natural voluntary movements that do not require the participant to learn a new skill, while Hocherman uses computer-generated movements which have to be learned and tracked by the patient. In contrast to the '602 Gierhart & Kopp patent, the present invention derives exact and multiple handwriting dysfluency measures, while Gierhart & Kopp provide merely a qualitative feedback of dysfluency in their advanced computer-based handwriting instruction system.

In contrast to actometry, the present invention also records the movement at high temporal resolution.

The present invention is implemented for low cost, off-the-shelf hardware. Therefore, a commercial embodiment of the present invention is deployable without specially sourced equipment and without hardware-intensive technical assistance.

When the present invention becomes routinely applied by psychiatrists, they will be able to combine it with their conventional ratings. This equips the psychiatrist with options for early detection of longitudinal changes in a patient's movements. Once FDA approval has been obtained, the present invention can be used by the psychiatrists to guide clinical decisions when managing medication-induced movement problems. This in turn will help the patients by making it less likely that they will be prescribed medication that could harm them in the long term.

The present invention enables health care professionals to measure and immediately evaluate movement side effects due to medications in psychiatric and neurological patients. This will help health professionals to select appropriate drugs and dosages for their patients. Eventually, this will help to protect patients from the permanent brain damage of long-term use of inappropriate medications or excessive dosages of medications. The invention will enable psychiatrists and neurologists to offer better and ultimately more cost-effective health care.

Neuroleptic medication side effects exemplify a problem the present invention solves. Many other medications and conditions exist influencing movement control. This will provide opportunities to benefit from the present invention.

Unlike traditional visual observation-based severity ratings, instrumental systems are able to measure movement abnormalities along a linear continuum of severity.

Because of their superior sensitivity, instrumental assessments can reveal subtle motor abnormalities below the threshold of detection by clinical evaluation, thus, creating the potential to reveal even subclinical motor phenomena. Instrumental measures are more reliable than observer-based ratings scales because they do not depend on examiner experience and are not subject to examiner bias.

In summary, the present invention is a handwriting-based tool which can be used, among other applications, for EPS tests that can be applied in psychiatric practices both for individual patient monitoring and for large multi-site clinical trials. The measures of handwriting kinematics are demonstrated to be sensitive to type and dose of antipsychotic medication in schizophrenia patients and can be used for many other patients and drugs (Caligiuri et al., 2006; Caligiuri et al.).

### BRIEF DESCRIPTION OF DRAWINGS

FIG. 1 is a flow chart of an exemplary method of handwriting movement analysis in accordance with the present invention including a testing procedure with several tasks in fixed or random sequence and several trials in blocked or random sequence;

FIG. 2 is a flow chart of the exemplary method of FIG. 1, showing data flow from the recording of movement device to the reporting of results of analysis;

FIG. 3 is a flow chart of an exemplary method of handwriting movement analysis in accordance with the present invention showing low-pass-filtered time functions and frequency spectra;

FIG. 4 is a flow chart of an exemplary method of handwriting movement analysis in accordance with the present invention showing segmentation into strokes using vertical velocity zero crossings or absolute velocity minima including an example of use of vertical velocity zero crossings;

FIG. 5 is a flow chart of an exemplary method of handwriting movement analysis in accordance with the present invention showing extraction of stroke features including duration, size, peak velocity, and normalized jerk;

FIG. 6 is Table 1 as referenced in the written description, showing results of analyses of scores for subjects grouped according to antipsychotic treatment;

FIG. 7 is a graph of effects of medication type on ANJ showing means and 95th % confidence intervals, demonstrating that dysfluency, measured as average normalized jerk, discriminates between medications;

FIG. 8 is a graph of velocity scaling for patients grouped by type of antipsychotic medication, demonstrating that dysfluency, measured as velocity scaling, discriminates between different medications;

FIG. 9 is a graph of SAEPS scores for patients receiving different medications, demonstrating that no significant differences were found;

FIG. 10 is a graph of AIMS scores for patients of different medications, demonstrating that no significant differences were found;

FIG. 11 is Table 2 as referenced in the written description, showing means (and standard deviations) for the handwriting kinematic variables for three subject groups, namely, Risperidone-treated schizophrenia (SZ), Unmedicated SZ, and Healthy persons;

FIG. 12 is a graph of average normalized jerk in the sentence-writing task relative to daily dose of risperidone;

FIG. 13 is a graph of vertical size in the sentence-writing task relative to daily dose of risperidone

FIG. 14 is Table 3 as referenced in the written description, showing correlation coefficients (r) for relationship between MovAlyzeR parameters and daily dose of schizophrenia patients taking risperidone (mg/day) for the complex loop patters (cursive lleellee with vertical sizes 1, 2, or 4 cm);

FIG. 15 is a graph of absolute jerk of the secondary submovement with respect to Risperidone dosage;

FIG. 16 is a graph of relative time to peak vertical velocity with respect to Risperidone dosage;

FIG. 17 is a graph of peak vertical acceleration with respect to Risperidone dosage;

FIG. 18 is a graph of relative duration of primary submovement with respect to Risperidone dosage;

FIG. 19 is a graph of relative size of primary submovement with respect to Risperidone dosage;

FIG. 20 is a graph of frequency of secondary submovement with respect to Risperidone dosage;

FIG. 21 is a graph of average absolute velocity with respect to Risperidone dosage;

FIG. 22 is a graph of peak vertical velocity with respect to Risperidone dosage;

FIG. 23, Table 4 as referenced in the written description, compares different patients with low- and high-dosage Risperidone using Student's t-test for unpaired data and averages of various parameters;

FIG. 24 is Table 5 as referenced in the written description, showing analysis of variance (“ANOVA”) in duration of secondary submovement across patients grouped according to medication type;

FIG. 25 is Table 6 as referenced in the written description, showing ANOVA in vertical size of secondary submovement across patients grouped according to medication type;

FIG. 26, Table 7, in similar manner shows ANOVA in vertical velocity of secondary submovement;

FIG. 27, Table 8, in similar manner shows ANOVA in vertical acceleration of secondary submovement;

FIG. 28, Table 9, in similar manner shows ANOVA in relative time to peak velocity of secondary submovement;

FIG. 29, Table 10, in similar manner shows ANOVA in absolute size of secondary submovement;

FIG. 30, Table 11, in similar manner shows ANOVA in absolute velocity of secondary submovement;

FIG. 31, Table 12, in similar manner shows ANOVA in road length of secondary submovement;

FIG. 32, Table 13, in similar manner shows ANOVA in absolute jerk of secondary submovement;

FIG. 33, Table 14, in similar manner shows ANOVA in number of peak acceleration points per stroke of secondary submovement;

FIG. 34, Table 15, in similar manner shows ANOVA in relative duration of primary submovement;

FIG. 35, Table 16, in similar manner shows ANOVA in relative size of primary submovement;

FIG. 36, Table 17, in similar manner shows ANOVA in frequency of secondary submovement per stroke;

FIG. 37, Table 18, in similar manner shows ANOVA in average normalized jerk of total movement;

FIG. 38 shows a chart of duration relative to type of medication;

FIG. 39 shows a chart of duration relative to task and type of medication;

FIG. 40 shows a chart of vertical size relative to type of medication;

FIG. 41 shows a chart of vertical size relative to task and type of medication;

FIG. 42 shows a chart of peak vertical velocity relative to type of medication;

FIG. 43 shows a chart of peak vertical velocity relative to task and type of medication;

FIG. 44 shows a chart of peak vertical acceleration relative to type of medication;

FIG. 45 shows a chart of peak vertical acceleration relative to task and type of medication;

FIG. 46 shows a chart of relative time to peak vertical velocity relative to type of medication;

FIG. 47 shows a chart of relative time to peak vertical velocity relative to task and type of medication;

FIG. 48 shows a chart of absolute size relative to type of medication;

FIG. 49 shows a chart of absolute size relative to task and type of medication;

FIG. 50 shows a chart of average absolute velocity relative to type of medication;

FIG. 51 shows a chart of average absolute velocity relative to task and type of medication;

FIG. 52 shows a chart of road length relative to type of medication;

FIG. 53 shows a chart of road length relative to task and type of medication;

FIG. 54 shows a chart of absolute jerk relative to type of medication;

FIG. 55 shows a chart of absolute jerk relative to task and type of medication;

FIG. 56 shows a chart of number of peak acceleration points relative to type of medication;

FIG. 57 shows a chart of number of peak acceleration points relative to task and type of medication;

FIG. 58 shows a chart of relative size of primary submovement relative to type of medication;

FIG. 59 shows a chart of relative size of primary submovement relative to task and type of medication;

FIG. 60 shows a chart of relative duration of primary submovement relative to type of medication;

FIG. 61 shows a chart of relative duration of primary submovement relative to task and type of medication;

FIG. 62 shows a chart of frequency of secondary submovement relative to type of medication;

FIG. 63 shows a chart of frequency of secondary submovement relative to task and type of medication;

FIG. 64 shows a chart of average normalized jerk per trial relative to type of medication; and

FIG. 65 shows a chart of average normalized jerk per trial relative to task and type of medication.

FIG. 66 shows a graph of vertical velocity with respect to time exemplifying the identification of a submovement;

FIG. 67 shows a graph of upstroke duration with respect to time during medication cycle in a medicated Parkinson's disease patient;

FIG. 68 shows a graph of stroke size with respect to stroke number in three classes of patients;

FIG. 69 shows a collection of writings including lops, “lleellee” pattern, circles, and a sentence;

FIG. 70 shows a template used in accordance with the present invention with instructions to write eight loops in the top writing area (small size scale) and loops written in that area;

FIG. 71 shows a template used in accordance with the present invention with instructions to write a sentence in the middle writing area (medium size scale) and a sentence written in that area;

FIG. 72 shows a chart of velocity scaling slope with respect to medication group (with means and 95% confidence intervals); and

FIG. 73 is Table 18 as referenced in the written description, listing handwriting measurement features.

### DESCRIPTION OF EMBODIMENTS

### Exemplary Procedures

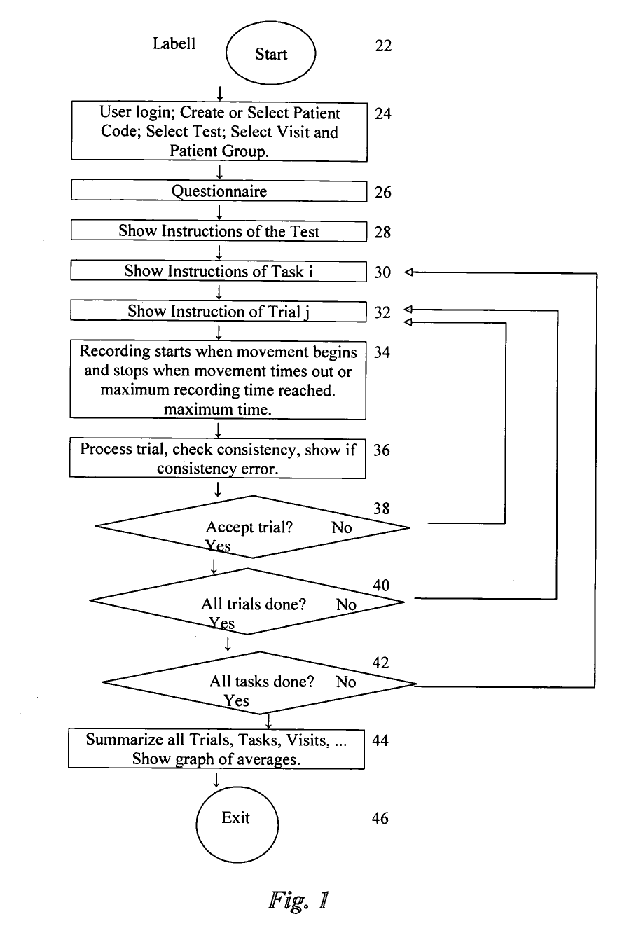

With reference to FIG. 1, a flow chart of an exemplary method of handwriting movement analysis in accordance with the present invention including a testing procedure with several tasks in fixed or random sequence and several trials in blocked or random sequence, a computer-implemented testing procedure collects reported information and raw physical data inputs. Start condition 22 leads to a registration protocol 24 during which data recorded to identify the person administering the test (“user”), the patient, any group the patient is regarded as belonging to, the test being administered, and the time of the test. Thereafter, a questionnaire 26 guides the user to record additional information about the patient including, for example, the patient's history, diagnosis, medication status, symptoms reported, and signs observed. Before testing begins, the patient is shown instructions, show-test-instructions 28, for completing the test.

The test procedure continues to show-task-i-instructions 30, a display of instructions to the user and also to the patient to cue the patient in how to perform a handwriting task numbered i, where i is a counter associated with the task, there being other tasks in the test. Next, at show-trial-j-instructions 32, instructions are displayed to the user and also to the patient. The user can then instruct the patient to start writing the next pattern at a particular place on the digitizer. This performance is numbered j, where j is a counter and more trials will follow immediately.

At recording starts 34, when the patient begins to move the writing implement, the movement is detected and recording begins. Recording stops when movement stops or when a predetermined maximum recording time is exceeded. At process trial 36, the recording is checked for consistency with the instructions the patient received at show-task-i-instructions 30. The consistency test includes verifying that the patient performed the 1-loops and the correct pattern of relative sizes.

At accept trial 38, a decision is made based on the consistency result of the analysis at process trial 36. If the consistency result did not exceed a predetermined value, then the trial is rejected and control passes back to show-trial-instruction-j 32 with the same value of j for a repeat of the trial. If the consistency result exceeded the predetermined value, then the trial is accepted, j is incremented and control passes to all trials done 40, where the value of j is incremented tested to determine whether the patient has completed enough trials of the task. If more trials are needed, control returns again to show-trial-j-instruction 32 with j incremented. If enough trials are complete, i is incremented and control passes to all tasks done 42, where the value of i is tested to determine whether the patient has completed all tasks in the test. If more tasks remain to be completed, then control returns to show-task-i-instruction with i incremented. If the patient has completed all of the tasks, then control passes to summarize 44, where a summary of the patient's trials, tasks and visits is displayed to the user and the testing procedure ends at exit 46.

The above testing procedure may be summarized as follows: during a patient's visit, the procedure collects reported information via standard computer inputs and collects raw physical data with a handwriting recording device. The system records raw data on the patient's performance of each of one or more tasks, and for each task prompts the patient to perform one or more trials of that task, each time, checking the data to ensure that the performance was consistent with the instructions.

FIG. 2 is a flow chart of the exemplary method of FIG. 1, showing data flow from the recording of movement device to the reporting of results of analysis. At start writing 48, the input device 50 is in communication with the personal computer 52, which includes a device driver and application interface providing real-time display feedback to the patient, receives pen coordinates x, y, z, and sends settings and queries.

The personal computer 52 stores raw device data 54 as a .hwr file of position (x,y) and axial pen pressure.

Time functions module 56 (“TIMFUN”) includes low-pass-filtered and differentiated time functions and frequency spectra. Time functions module 56 receives raw data 54 and produces and stores processed data 58 as a .tf file of filtered x, y; raw z, x, y; absolute velocity, acceleration, jerk, and frequency spectra.

At segmentation 60 (“SEGMEN”), processed data 58 are received and vertical velocity zero crossings or absolute velocity minima are used to segment the data into strokes, which are stored as a .seg file of segmentation points 62 including submovement segmentation points.

Extraction 64 includes extraction of stroke features such as duration, size, peak velocity, and normalized jerk. The inputs are processed data 58 and segmentation points 62. The result is stored as an .ext file of extracted features per stroke 66 for all trials of the task condition. In our psychomotor tests, a condition is defined as a specific writing pattern performed in a specific way. For example, a writing pattern performed at 1 cm height is a different condition than the same writing pattern performed at 2 cm height.

Consistency checking 68 (“CONSIS”) receives extracted features per stroke 66 and stores a .con file of consistent extracted features per stroke 70 for all trials of the condition.

Summarize trials 72 (“SUMTRIAL”) summarizes selected patient and feature data, receiving consistent extracted features per stroke 70 and storing an .inc file of consistent extracted summarized data 74 for all consistent data.

Analyze all data 76 (“SHOWSTATS”) produces graphic, numerical and statistical renderings of the data and computations, receiving consistent extracted summarized data 74 and producing and storing a .tmp analysis file 78 for all consistent transformed data. A report 80 is generated in a form convenient for use in an evaluation step 82.

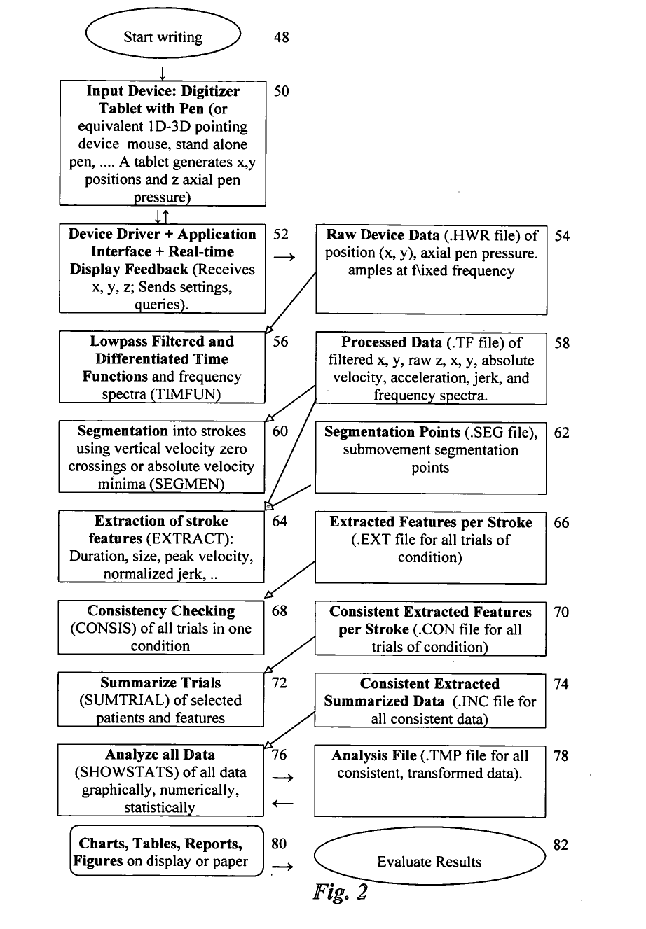

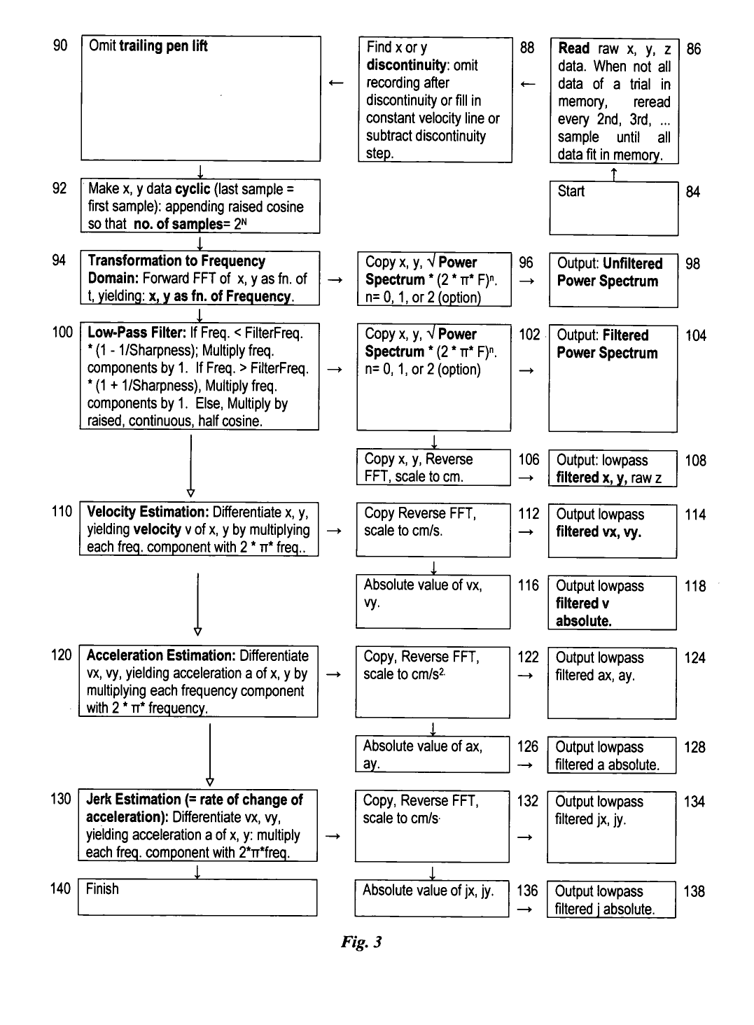

The time functions module 56 is described in greater detail in FIG. 3, a flow chart of an exemplary method of handwriting movement analysis in accordance with the present invention showing low-pass-filtered time functions and frequency spectra, beginning at time function start 84.

At read 86, raw x, y, z data are read into memory. For long recordings and slow movements, only every second, third, . . . sample is read into memory.

Sometimes, patients may slightly lift the pen during writing (especially when writing a sentence). Pen movements will be recorded by the digitizers even while the pen is lifted slightly off the paper. However, if the pen is lifted higher than some tablet-specific maximum pen-lift height, the digitizer will not deliver samples until the pen is again in proximity of the tablet. When the pen is lowered at another spot the sequence of samples will make a sudden jump. This is called a discontinuity. Our digital signal analysis would show artifacts at those discontinuities. Therefore, at discontinuity 88, a discontinuity is found in x or y and is used as a trigger either to omit processing after the discontinuity or to fill in a constant velocity line or to shift all subsequent samples so that

At omit trailing pen lift 90, the trailing pen lift is omitted. While brief pen lifts during a writing task are common and will be left intact to maintain continuity, pen lifts at the end of a writing pattern are recorded until the pen-lift period exceeds the end-of-recording time out (for 1-3 s). This trailing pen lift is not required to maintain continuity, therefore, it can be omitted.

At cyclic 92, the x, y data are made cyclic (value of last sample equal to value of first sample) by appending a raised cosine so that the number of samples can be expressed as a power of 2, thereby satisfying the continuity requirement for applying the fast Fourier transformation of the data.

At transformation to frequency domain 94, a forward fast Fourier transform (FFT) of x and y as a function of time yields x and y as a function of frequency. The outputs of this step include a power spectrum 96 and an unfiltered power spectrum 98. Additionally, the expression of x and y as a function of frequency is passed to a low-pass filter 100.

Low-pass filter 100 implements a three-way decision. If frequency is less than filter frequency multiplied by 1−(1/sharpness) or greater than filter frequency multiplied by 1+1/sharpness, then frequency components are multiplied by 1. Otherwise, they are multiplied by a raised, continuous, half cosine. The outputs of this step lead to another copy of power spectrum 102 and filtered power spectrum 104, creation of a reverse FFT 106, and output of low-pass-filtered x and y with raw z data. Additionally, the output is passed to velocity estimation 110.

Velocity estimation 110 differentiates x, y data to yield velocities of x and y. This is done by multiplying each frequency component with 2*π*frequency. The outputs of this step lead to creation of a reverse FFT 112 scaled to centimeters per second, output of low-pass-filtered velocities 114, absolute velocities 116, and low-pass-filtered absolute velocities 118. Additionally, the output is passed to acceleration estimation 120.

Acceleration estimation 120 differentiates vx, vy data to yield accelerations of x and y. This is done by multiplying each frequency component with 2*π*frequency. The outputs of this step include reverse FFT 122 scaled to centimeters per second squared, low-pass-filtered accelerations ax and ay 124, absolute accelerations 126, and low-pass-filtered absolute accelerations 128. Additionally, the output is passed to jerk estimation 130.

Jerk estimation 130 differentiates ax, vy data to yield jerk of x and y. This is done by multiplying each frequency component with 2*π*frequency. The outputs of this step include reverse FFT 132 scaled to centimeters per second raised to the third power, low-pass-filtered jerk jx and jy 134, absolute jerks 136, and low-pass-filtered absolute jerks 138, after which step the time function has reached its finish 140.

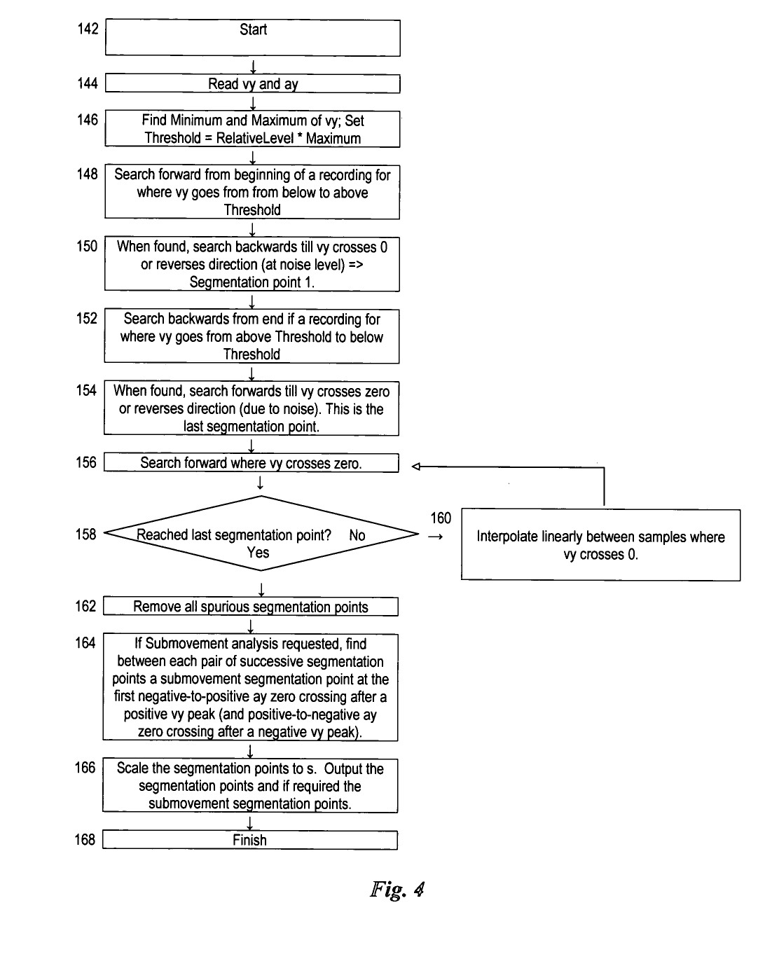

Segmentation 60 is described in greater detail in FIG. 4, a flow chart of an exemplary method of handwriting movement analysis in accordance with the present invention showing segmentation into strokes using vertical velocity zero crossings or absolute velocity minima including an example of use of vertical velocity zero crossings.

After segmentation start 142, vertical velocity vy and vertical acceleration ay are read at read 144 and these data are passed to a series of steps for determining segmentation points.

At step find 146, a procedure finds a minimum and maximum of vy; a threshold of, for example, 0.1 times relative to the absolute extreme is set.

At step search forward 148, a procedure searches forward from the beginning of a recording for a crossing of vy from below the threshold to above it. From this crossing, a procedure at step search backwards 150 searches backwards until vy crosses 0 or reverses direction (at noise level); this crossing is designated the beginning of the pattern which is also the first segmentation point.

At step search backwards from end 152, a procedure searches backwards from the end of the recording for a crossing of vy from above the threshold to below it. From this crossing, a procedure at search forward 154 searches forward until vy crosses zero or reverses direction due to noise. This crossing is designated the last segmentation point.

At step search forward 156, a procedure again searches forward for where vy crosses zero.

At reached last 158, a decision is made. If the last segmentation point has not been reached, then at interpolate 160 a procedure interpolates linearly between samples where vy crosses zero and control step search forward 156 is repeated. If the last segmentation point has been reached, then at remove 162 all spurious segmentation points are removed.

At step submovement 164, if submovement analysis has been requested, a procedure finds between each pair of successive segmentation points a submovement segmentation point at the first negative-to-positive ay zero crossing after a positive vy peak, and likewise, the first positive-to-negative ay zero crossing after a negative vy peak.

At step scale 166, the segmentation points are scaled and outputted. Submovement segmentation points are included in the output if required. The segmentation process is at its finish 168.

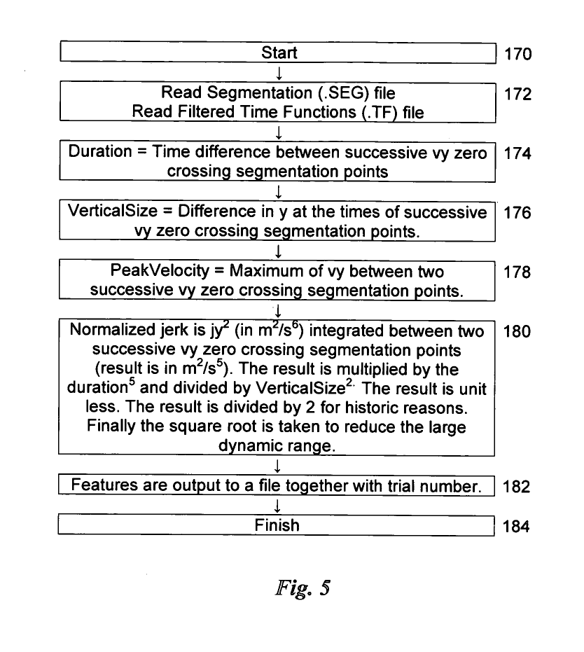

Extraction of stroke features 64 is described in greater detail in FIG. 5, a flow chart of an exemplary method of handwriting movement analysis in accordance with the present invention showing extraction of stroke features including duration, size, peak velocity, and normalized jerk, beginning at extraction start 170.

At read 172, a .seg segmentation file and a .tf filtered time functions file are read. At duration 174, duration is computed as a time difference between successive vy zero crossing segmentation points.

At vertical size 176, vertical size is computed as a difference in y at those segmentation points.

At peak velocity 178, peak velocity is computed as the maximum of vy between two successive vy zero crossing segmentation points.

At normalized jerk 180, normalized jerk, the product of jerk and the second power of y, in units of meters squared over seconds to the sixth power, is integrated between two successive vy zero crossing segmentation points, the result being in units of meters squared over seconds to the fifth power. This result is divided by 2 for historic reasons. Its square root is taken in order to reduce the dynamic range.

At output 182, the features are displayed or communicated along with a trial number identifying the trial (as previously discussed, the patient is asked to perform several trials of each task). The extraction process is at its finish 184.

### Movement Measurement Devices

The patient's movements are measured using an off-the-shelf writing tablet or graphical digitizer device or an equivalent system that records the position of a hand-held pen or other object. The device precisely records movements in one or two dimensions. Additionally, it records position or force components in the axial direction of the pen. Thus, when the pen is in the air, pen pressure is small or 0. If the pen is pressed on the writing surface like during a normal act of writing, the axial pen pressure is higher. Instead of a digitizer, other devices can be used as well as long as they record at least one movement dimension at a high, fixed frequency. Examples of those other devices include but are not limited to a computer mouse, free standing pen with acoustic or electronic remote sensing, pen with built in accelerometers, single or multiple-optical camera, or electromagnetic sensors. All these devices have in common that they allow 1-3 dimensional position recording at a sufficiently frequency (e,g., much higher than the minimum bandwidth of 20 Hz which is 2 times the highest frequency). To prove they record the movement in time and space the data should allow the exact movement to be replayed. Different devices will have different specifications. Good devices achieve x and y accuracies of 0.01 cm and 100-200 Hz sampling rate and sample the x and y position simultaneously and at a fixed rate.

### Movement Description: Static

Various ways exist to describe a movement of the “effector endpoint” such that the movement can be replayed in relative space and relative time. The effector endpoint is the for example, the position of the mouse cursor, or the position of the pen tip. In general, it is the point that the human movement system is controlling in some way. Without limiting the invention we state that one way to describe such an effector endpoint movement consists of a sequence of x coordinates with known relative intervals so that an accurate x-versus-(relative)-time curve can be produced. Another example of a movement representation is a sequence of (x,y,p) coordinates recorded at a fixed, known frequency, where x and y are the x and y coordinates and p is the axial pen pressure. Another example of a movement recording is a sequence of (t, x, y, z, p, r, alpha, beta, gamma) where t is a time stamp, x, y, z are the positions in the x,y,z space, p is the axial pen pressure, r is the rotation of the pen barrel, alpha and beta are the angles of between the pen barrel and the (x,y), (y,z), and (x,z) planes, respectively (Gierhart & Teulings, 1995). Gierhart's system was designed to provide qualitative feedback about pen grip and movement fluency to children on how to learn to write (U.S. Pat. No. 5,730,602).

### Movement Description: Dynamic

The movement data or “time series” can be processed in a digital or analog computer. Various algorithms or wiring schedules exist that that can reduce noise in the recorded data. Fast movements have a peak frequency at 5 Hz while the maximum frequency found in the average movement frequency spectrum is about 10 Hz (Tailings & Maarse, 1984). These are the signal frequencies. The recording equipment noise has a broader frequency spectrum and can even be white (i.e., all frequency components are equally likely). These are the noise frequencies. Therefore, the higher noise frequencies can be removed while leaving the lower signal frequencies untouched by a low-pass or smooth filter. These low-pass filters are well known in analogue data processing. These low-pass filters can also be implemented in digital data processing. However, the sampling frequency of the end effector position should be twice the highest frequency (of signal and noise) or frequency aliasing will result. Thus, a weak analogue low-pass (anti-alias) filter would still be needed (as used when digitizing physiological signals). Therefore, when the anti-alias filter suppresses all frequencies above 10 Hz, a sampling frequency of 20 Hz could be sufficient in theory. However, when the data generated are immediately digitized (as in all digitizing tablets) an anti-alias filter cannot be inserted anywhere. Therefore, a higher than needed sampling rate must be used (e.g., 100-200 Hz). This allows for low-pass filtering, suppressing a large range of high-frequency noise components.

### Collecting the Handwriting Samples

The subjects are instructed what to write specific handwriting-like patterns on an off-the shelf digitizer tablet using the tablet's electronic pen. The tablet samples to position of the pen tip at an RMS accuracy of 0.01 cm and a fixed frequency (of 100-200 Hz) which needs to be several times above twice the typical bandwidth of handwriting movements (about 10 Hz).

The handwriting patterns consist of 8 continuous, overlapping circles, or 8 cursive 1-loops, or a cursive pattern of writing the letter sequence “lleellee.” Therefore, each of these patterns has 16 up or down strokes. The patient is instructed to not count the loops. Instead, the person administering the test to the patient will see a real-time stroke counter and will tell the patient when to stop writing.

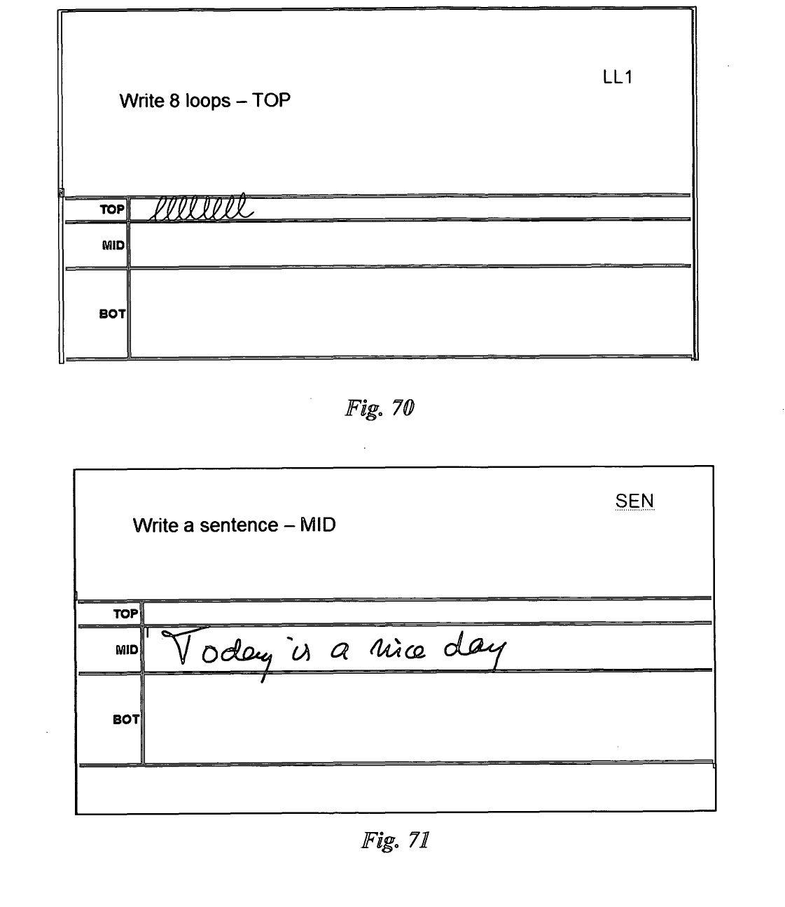

FIG. 69 shows a collection of writings of the type requested in accordance with the present invention, including lops, “lleellee” pattern, circles, and a sentence. FIG. 70 shows a template used in accordance with the present invention with instructions to write eight loops in the top writing area (small size scale) and loops written in that area. FIG. 71 shows a template used in accordance with the present invention with instructions to write a sentence in the middle writing area (medium size scale) and a sentence written in that area. Thus, it will be appreciated that the instructions given on the template, in combination with the size of the writing space the instruction directs the patient to write in, can guide the patient with little or no intervention from the user who is administering the test, thereby reducing the likelihood of user-induced bias.

The real-time stroke counter uses a simple algorithm that looks for samples with lower and lower y values and remembers the lowest y value until a sequence of higher and higher y-values is received.

The recording of a trial starts as soon as the pen touches the tablet. When the patient stops and lifts the pen, after a time out, the recording ends. The raw data will be stored and be processed immediately. Processing includes a consistency check explained elsewhere in this description. The user will have the opportunity to view the cause of the consistency failure and to decide to instruct the patient how to redo the trial.

### Noise Filtering

A simple example of digital low-pass filtering is averaging the previous, the current, and following samples to yield a new sample. More sophisticated low-pass filters exist, such as convolution windows, Butterworth filters, frequency domain filters. We now explain in greater detail the use of a frequency-domain filter. Many transforms exist that translate a time function in a sort of spectrum of lower or higher frequency components. One example is the Fast Fourier Transform (FFT). The inverse transform would restore the time signal. By suppressing the higher frequency components before the inverse transform, the time function will be much smoother as the small ripples have been removed. The FFT is subject to the following restrictions: (1) the number of samples must be a power of 2; and (2) the signal must be continuous and cyclical so that the end and the beginning of the signal must be the same. These conditions are satisfied by appending a sine wave after the end point of a recording that brings the time function back to the first value while adding sufficient samples so the net number of samples becomes a power of 2.

### Time Derivatives

Various algorithms exist to estimate time derivatives (e.g., the speed of change with time). The first time derivative is the velocity as a function of time. The second time derivative is the time derivative applied on the first time derivative, yielding the acceleration as a function of time. The acceleration is proportional to the force in the friction-less approximation. The third time derivative is the first time derivative applied 3 times yielding the jerk as a function of time. Jerk represents the change of force.

### Estimating Time Derivatives

Various algorithms exist to differentiate time functions. A simple example to differentiate data is the difference between previous and following sample. Another example of differentiation is to multiply each frequency component with 2*pi*frequency (where pi=3.1418 . . . ). This works because the time derivative of sin (2*pi*frequency*time)=2*pi*frequency cosine (2*pi*frequency*time). Thus, it is possible to reliably estimate position, velocity, acceleration, and jerk as a function of time.

### Segments

The next stage is to segment the time functions into functional movement units. Several examples of those functional units exist. An example of segmentation is based on absolute velocity minima. From velocity minimum to velocity minimum is a ballistic movement segment with one peak velocity. We now describe another example of a segmentation that works well in Western cursive handwriting: upward and downward strokes. Segmentation is possible using zero crossings of the vertical velocity component. The advantage is that, by using linear interpolation, the zero crossing can be identified with smaller uncertainty than half the inter-sample-period (inverse of the sampling rate). In non-Western handwriting systems other patterns and segmentations exist.

### Segmentation

An example of a writing pattern that is easily segmented is a sequence of 1-loops. The number of loops is not relevant. The number of loops should be about equal for all patients and not cause too much tiredness and fit inside the recording window. We tested with 8 loops, but any other number would work. This pattern can be performed at different sizes and instructions and does not require much practice.

### Feature Extraction

The next stage is feature extraction based on each segment (independently of a particular definition of the stroke). Many algorithms exist expressing certain features of the human motor system. Several very different algorithms exist expressing essentially one underlying motor control feature of the human motor system. We list here the features that are relevant for this invention:

### Handwriting Movement Measures

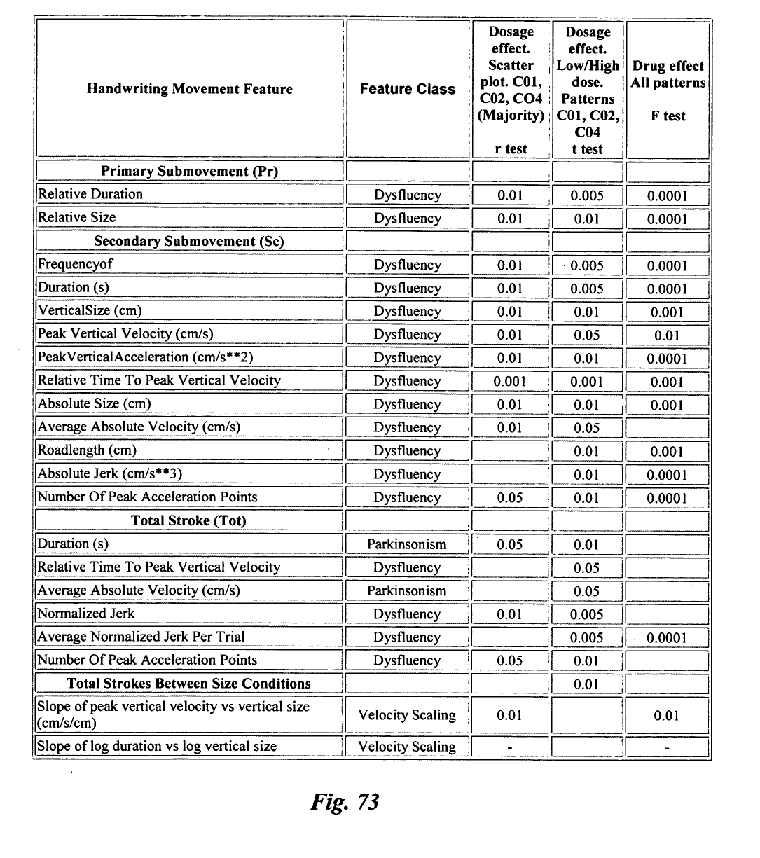

With reference to FIG. 73, Table 18 lists handwriting measurement features and classifies them according to how they are derived and also according to how they are used in at least one embodiment of the present invention. The system generates 3 classes of handwriting movement features: Dysfluency, Velocity Scaling; and Bradykinesia.

### Dysfluency

Measures expressing dysfluency include the following:
1. Normalized jerk of total stroke
2. Average normalized jerk (=Normalized jerk per stroke averaged across ALL strokes produced in a trial, even if more strokes are produced than required)
3. Relative Duration of primary submovement (DECREASES with increasing dysfluency)
4. Relative Size of primary submovement (DECREASES with increasing dysfluency)
5. Frequency of secondary submovements
6. Duration of secondary submovement
7. Vertical Size of secondary submovement
8. Peak Vertical Velocity of secondary submovement
9. Peak Vertical Acceleration of secondary submovement
10. Relative Time To Peak Vertical Velocity of secondary submovement
11. Absolute Size of secondary submovement
12. Road Length of secondary submovement
13. Average Absolute Velocity of secondary submovement
14. Absolute Jerk of secondary submovement
15. Number Of Peak Acceleration Points of secondary submovement
16. Relative Time To Peak Vertical Velocity of total stroke
17. Number Of Peak Acceleration Points of total stroke

Dysfluency expresses the number and amplitude of force adjustments during the performance of a movement pattern. Dysfluency is indicative of dyskinesias in patients. Highly practiced and fast patterns have low dysfluency while new and slow patterns have high dysfluency. Many algorithms for the dysfluency feature in a motor control exist. Without limiting the invention to a particular dysfluency measure we include several examples of dysfluency (Teulings et al., 2005).

A simple measure of dysfluency is the number of zero crossings of the acceleration (in y or x direction or any other direction), divided by the number of zero crossings of the velocity (Maarse et al., 1987). A sine wave is an example of a perfectly fluent movement. The velocity is then a cosine wave and the acceleration is then a sine wave. Cosine and sine waves have an equal number of zero crossings. Therefore the ratio is 1. With increasing dysfluency, the number of zero crossings of the acceleration increases while the number of zero crossings of the velocity will only increase in extreme conditions. Therefore the ratio will increase quickly to 3 or 5.

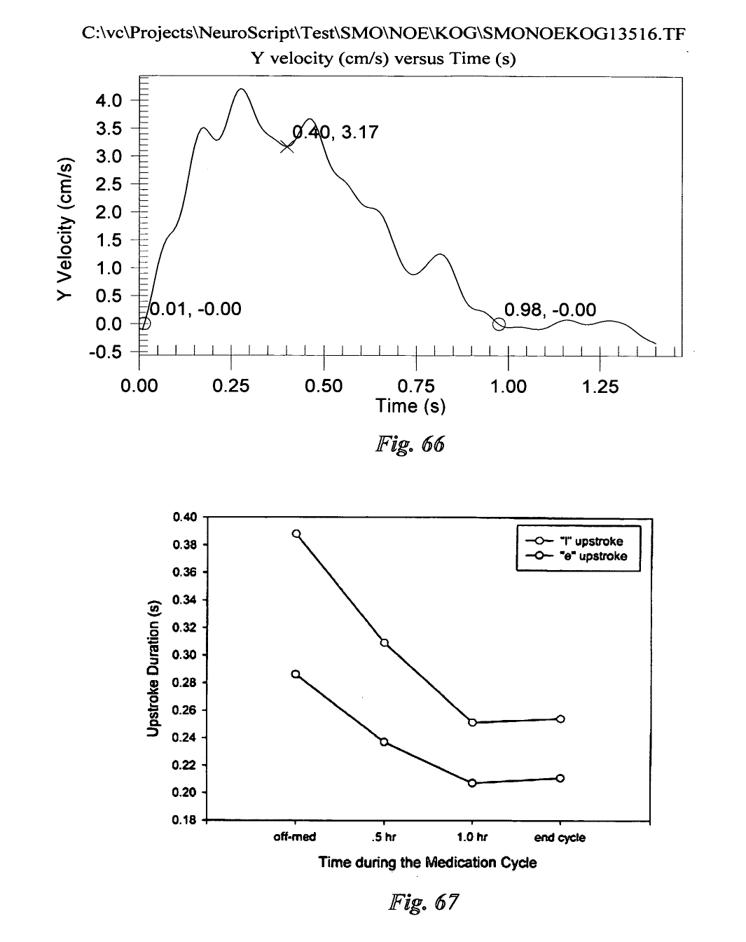

Another measure of dysfluency is based on submovement analysis (derived from Carlton, 1980; Crossman & Goodeve, 1963; Meyer et al., 1988; 1990; Walker, 1993; 1997). In submovement analysis each stroke is subdivided into a primary, ballistic movement that includes the velocity peak. A stroke can be a goal-directed movement from Point A to Point B, or a separate up-stroke or down-stroke in a repetitive loops or overlaying-circles writing pattern. Thus, during a stroke in a repetitive loops or circles pattern, the pen is targeting the upper guide line or the lower base line. The total movement is the entire, un-subdivided stroke. The primary submovement constitutes the first part of the stroke that contains the velocity peak. The part that contains the velocity peak is defined as the part that is primarily ballistic, as a preprogrammed movement. After the peak vertical velocity will decrease. When it would decrease towards zero at the end of the stroke, there would not be a secondary submovement and the primary submovement is equal to the total stroke movement. However, vertical velocity may also reach a relative minimum after the vertical velocity peak, and then increase again. This latter point can be mathematically identified as the FIRST (negative-to-positive) vertical acceleration zero crossing AFTER the vertical velocity peak (which is a positive-to-negative vertical acceleration, point). This point is defined as the end of the primary submovement and the beginning of the secondary submovement FIG. 66 shows a graph of vertical velocity with respect to time exemplifying the identification of a submovement. The two circles mark the beginning and end of a stroke at zero vertical velocity. The small x after the velocity peak marks the separation between primary and secondary submovements.

The first negative-to-positive acceleration zero-crossing after the peak is defined as the end of the primary submovement. All acceleration oscillations until the end of the stroke are defined as secondary submovements. In a perfectly smooth stroke the end of the primary submovement coincides with the end of the stroke. Therefore the relative duration and size of the secondary submovement (i.e., secondary submovement duration/total stroke duration) is 0. The relative frequency of secondary submovements across many of such perfectly smooth strokes will be 0 as well. With increasing dysfluencies, the relative duration and size of the secondary submovement increases from 0 until the theoretical maximum of 1.

The relative size, duration, and frequency of secondary submovement form three other measures of dysfluency (Teulings et al., 2005).

Yet another measure of dysfluency is the time and size to the moment of peak velocity relative to the total stroke time and size, respectively. In a perfectly smooth movement, this ratio is 0.5. Departures from this ideal value refer to departures from fluency.

A frequently-used dysfluency score is based on the jerk time function across a stroke. Jerk is the 3rd time derivative and expresses the amount of force changes during movements. To obtain one summary score for one stroke, jerk is squared and integrated across the entire stroke. Jerk increases with stroke size and decreases with the third power of duration. Therefore, normalization is required to be able to compare small and large movements and slow and fast movements.

To normalize, integrated, squared jerk (in units of meters squared over seconds to the fifth power) is multiplied with the fifth power of duration and divided by the second power of size. For reasons of compatibility the square root is taken of half of the result. Only the vertical jerk is used in our example. We can also use the horizontal jerk or the absolute jerk (Teulings et al., 1997).

All these dysfluency algorithms can be used and combined if needed to obtain an effective dysfluency quantification of the movement.

### Velocity Scaling

Two measures of velocity scaling fall into this class.

1) Slope of peak velocity versus size

2) Slope of relative duration versus relative size

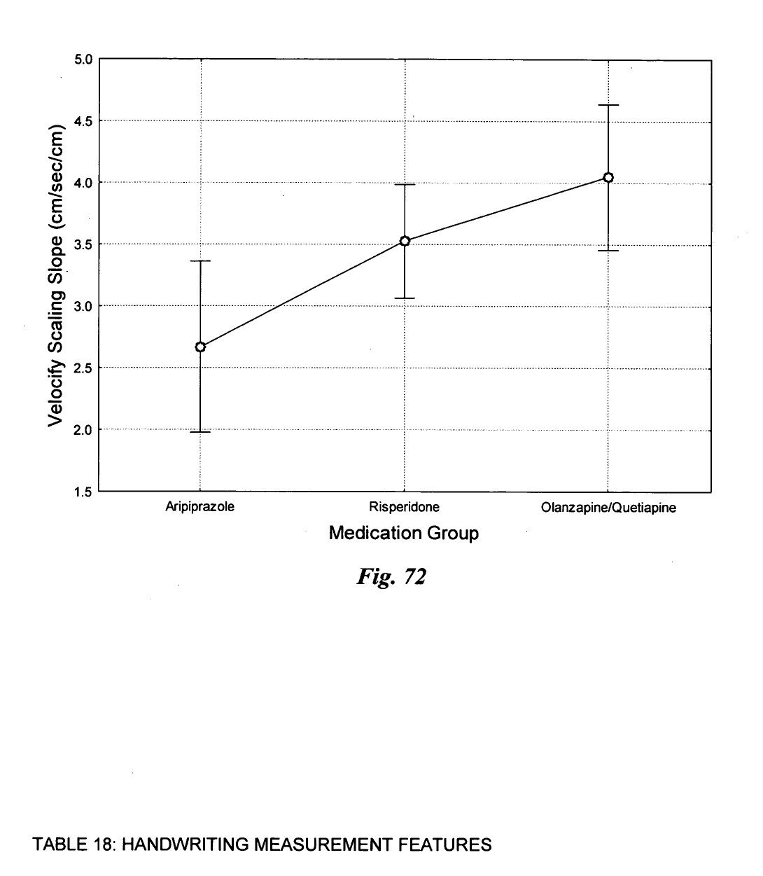

The first Velocity Scaling (VS) measure (i.e., slope of peak vertical velocity versus vertical size) changes significantly with medication type. With reference to FIG. 72, a chart of velocity scaling slope with respect to medication group, ANOVA shows significant effect of medication type on VS (F=4.5, df=2,200, p=0.01). Patients on aripiprazole had lower VS scores than those on olanzapine or quetiapine. Lower VS scores indicate more parkinsonian bradykinesia.

This measure expresses the extent to which movements are sped up when size is increased. This measure can be indicative for Parkinsonian bradykinesia, as these patients are unable to scale movements normally (Pfann et al., 2001). This accounts for the classic observation in Parkinson's disease that patients execute a variety of movements at the same speed. For handwriting measurement, strokes of different sizes can be acquired with a single recording (e.g., when using the example of a handwriting movement consisting of a more complex sequence of large and small loops, “lleellee”), or different sizes can be derived from different recordings where in one recording the movement size was instructed to be of one particular size and in another recording of another particular size. Without limiting the invention to these, several measures exist.

One simple and direct measure is the increase of a velocity measure in one stroke (e.g., in cm/s) divided by the increase of size (e.g., in cm). This resulting velocity scaling has a unit of size/duration/size or 1/duration.

Size of Movement (required to calculate this Velocity Scaling measure) is the 1-dimensional or 2-dimensional spatial extent or amplitude of the movement pattern. Examples of 1-dimensional extension measures include vertical size of a stroke, horizontal size of a stroke, beginning-to-end distance of stroke, and path length of a stroke. The 2-dimensional spatial extension measures include the product of horizontal and vertical sizes, and also include enclosed loop sizes or similar measures.

Another example of velocity scaling is the slope of relationship between duration versus size of the movement. The unit of this slope is s/cm. A unit-free measure is slope in the log Duration vs log Size chart:

Slope=(log(duration2)−log(duration1))/(log(size2)−log(size1)).

Therefore, slope=log(duration2/duration1)/log(size2/size1).

Duration1 and size1 cannot be 0. Size2 cannot be size1. This method requires a sizable difference between size2 and size1. Logarithms are unitless numbers. They are essentially ratios between values of equal units. The quotient of logarithms is also unitless.

If slope=0, velocity scaling is perfect. If slope=1, there is no velocity scaling at all, i.e., velocity remains constant for small and large movements. If slope=½, movements are at constant force and velocity only increases due to longer stroke durations.

### Bradykinesia

Bradykinesia (slowness of movement) is one of the motor signs of parkinsonism. Parkinsonism also encompasses micrographia (small movements), rigidity and tremor. The measures of this class include the following:

1) Duration of total stroke

2) Average Absolute Velocity of total stroke

Duration of movement is the time (e.g., in seconds) it takes for the motor system to produce a certain writing pattern. A simple algorithm to calculate duration is the time difference between the beginning and the end of one, several, or particular strokes within one movement pattern's recording. Durations of several strokes within one trial or across replications of the trial can be averaged to obtain a better duration estimate.

### Average Absolute Velocity

The velocity is a quotient between a size and a duration measure. For example, vertical size divided by duration. Special velocity measures include but are not limited to Peak (positive for negative) velocity or the Peak absolute velocity, or the maximum of the absolute velocity during a stroke.

### Consistency Checking.

After a trial has been processed, and the features have been extracted, the same extracted features can be used to verify that the trial is sufficiently consistent with the instruction. For example, the instruction is to produce at least 8 loops or 16 strokes. So the trial needs to contain at least 16 strokes. Furthermore, the loop surface is calculated. In a cursive “1”, the second stroke closes a counterclockwise (positive turning direction) loop. Therefore the second stroke has a positive loop surface. If that positive loop surface is lacking in Stroke 2, the trial will be deemed inconsistent.

If the pattern requires producing an “lleellee” sequence, the largest of the e-loops needs to be smaller than the smallest of the 1-loops.

### Results

### EXAMPLES

### Example 1

Two studies were completed comparing handwriting quantification with traditional visual-based severity ratings (Caligiuri et al., 2006; Caligiuri et al, submitted). In these studies, three visual observation-based severity scales are used:
1) The Simpson-Angus EPS scale (SAEPS) to measure Extrapyramidal Side effects including parkinsonism.
2) The Abnormal Involuntary Movement Scale (AIMS) to measure Tardive Dyskinesia (TD)
3) The Barnes Akathisia Rating Scale (BARS) to measure akathisia.

The handwriting kinematic measures used for comparison included a measure of handwriting dysfluency (average normalized jerk, ANJ) and velocity scaling (VS). Normalized jerk scores were derived from the vertical movement component averaged over all trials in 9 handwriting tasks: overlaying continuous circles, left-to-right continuous loops, and complex loops (lleellee) with the dominant hand for the 1 cm, 2 cm, and 4 cm size conditions. The Velocity Scaling (VS) score was based on the peak velocity increase divided by the vertical size increase for trials of 1 cm 2 cm and 4 cm loops.

Subjects were selected for study based on their prescribed medications. There were four groups of subjects each treated with a different antipsychotic medication: aripiprazole, risperidone, olanzapine, or quetiapine. A group of healthy subjects was included for comparison purposes.

### Group Effects

The mean ANJ score for 9 task conditions for the schizophrenia patients was 45.0 (±87.4), where as the mean ANJ score for the healthy subjects was only 14.1 (±12.2). This difference was highly statistically significant (F_{1,881}=50.89; p<0.0001) and indicates that schizophrenia patients performed the writing tasks with higher levels of dysfluency than healthy subjects. Similarly, the mean VS score for 3 task conditions for the schizophrenia patients was 3.8 cm/s/cm (±2.2 cm/s/cm), where as the mean VS score for the healthy subjects was 4.6 cm/s/cm (±2.3 cm/s/cm). This difference was statistically significant (F_{1,296}=9.46; p<0.005) and indicates the presence of mild parkinsonian bradykinesia in the handwriting performance of schizophrenia patients.

### Sensitivity and Specificity

We examined the degree to which the MovAlyzeR variables accurately classified patients as normal or abnormal based on standard clinical rating scales. Two analyses were performed: one for upper extremity TD (based on AIMS ratings) and another for upper extremity bradykinesia (based on Simpson-Angus EPS ratings). Cut-points for classifying individuals into normal and abnormal groups were based on the 95% confidence intervals for the normal means. Results include the following:

1) ANJ indicated 81% (21/26) accuracy in classifying patients with at least mild upper extremity tardive dyskinesia (TD) (sensitivity).
2) VS indicated 68% (17/25) accuracy in classifying patients with at least mild upper extremity bradykinesia (sensitivity).

### Medication Type Effects

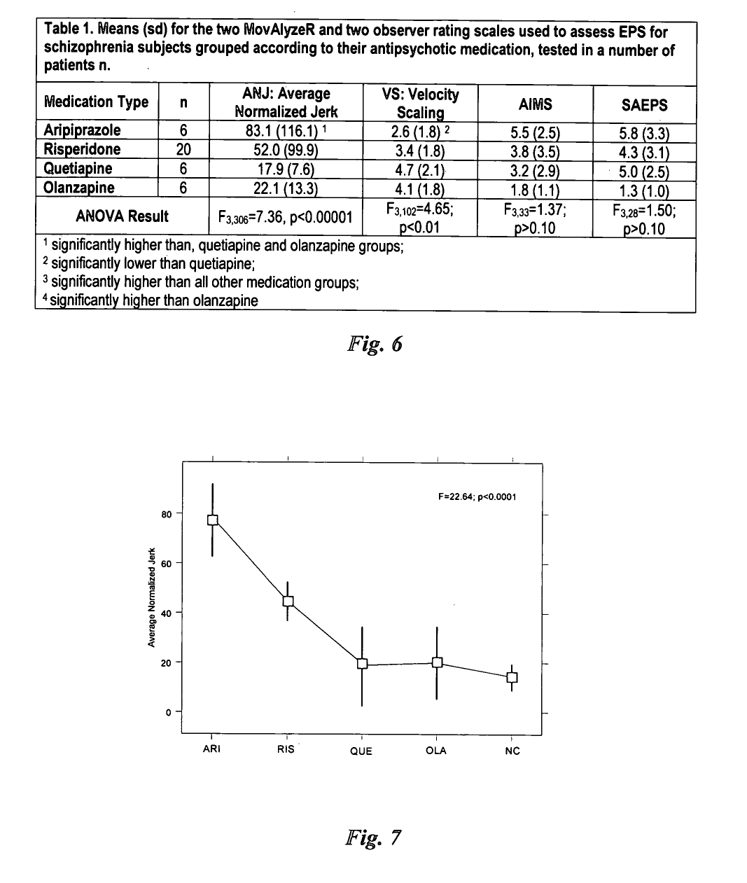

FIG. 6 contains Table 1, which shows the results of analyses of MovAlyzeR scores for subjects grouped according to antipsychotic treatment. Patients treated with aripiprazole exhibited significantly higher ANJ and lower VS scores than those on other medications. Patients taking quetiapine or olanzapine did not differ from normal healthy subjects on the ANJ measure, whereas, patients taking aripiprazole or risperidone had significantly higher ANJ scores than healthy subjects. FIG. 7 shows the variability in ANJ mean score across the 4 medication groups including aripiprazole (ARI), risperidone (RIS), quetiapine (QUE) and olanzapine (OLA) as well as the mean ANJ score for normal comparison subjects (NC). Results include:

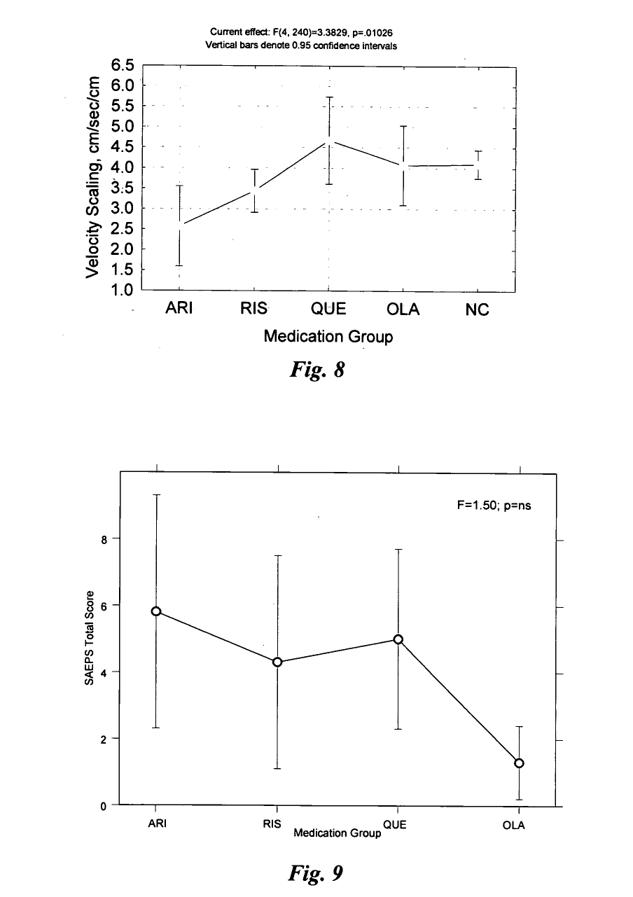

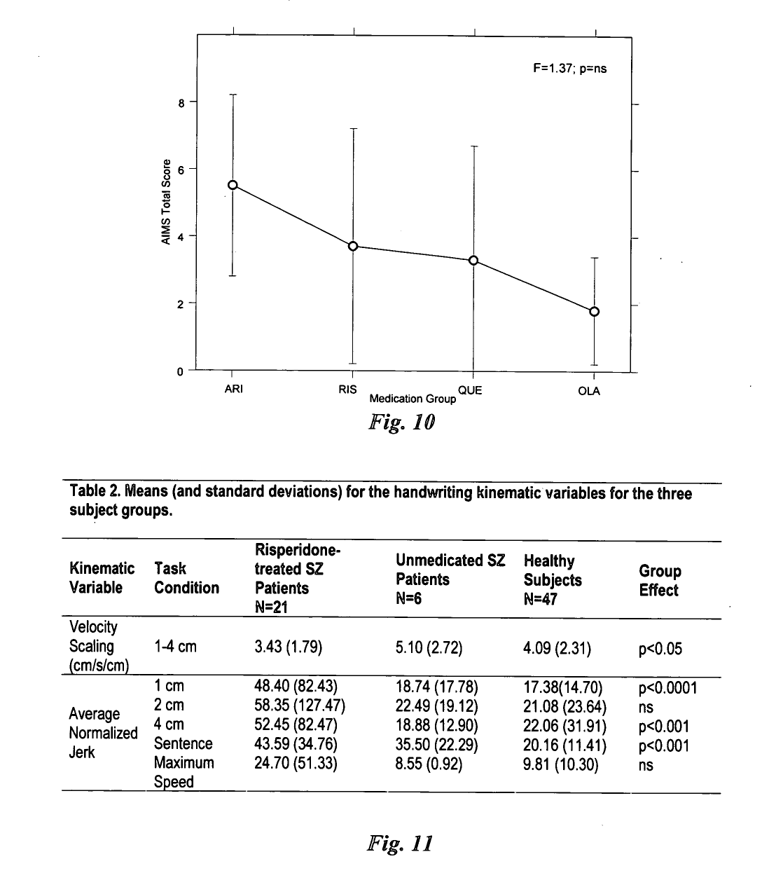

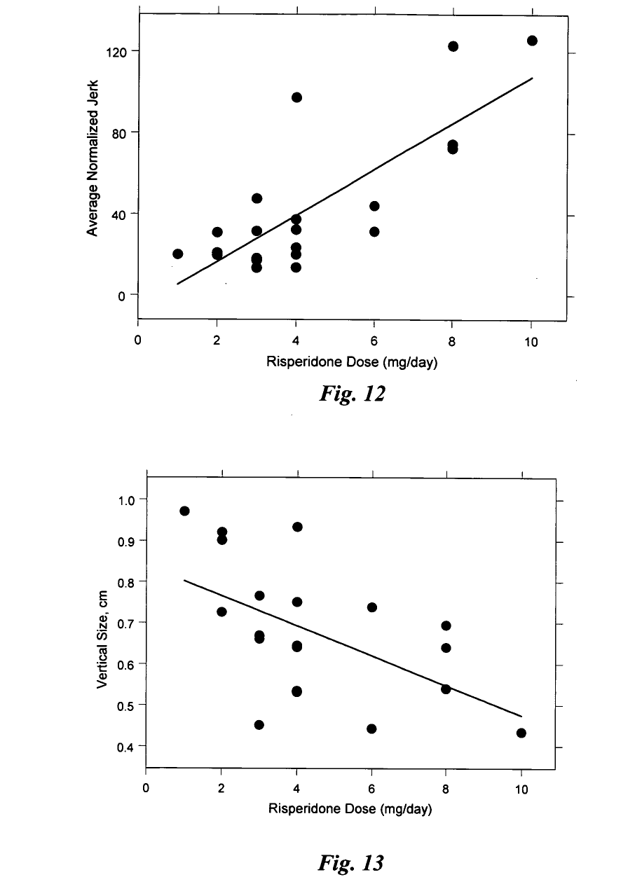

1) ANJ showed a significant group effect for risperidone versus quetiapine (Table 1 and FIG. 7).
2) VS (characterizing the extent of parkinsonism) showed significant differences between mediations (See FIG. 6 containing Table 1; see FIG. 8).
3) Conventional SAEPS and AIMS score failed to show a significant medication type relationship (See FIG. 9 and FIG. 10).

### Medication Dosage Effects

In the second study (Caligiuri et al., submitted), we examined whether the daily dose of risperidone results in changes in the observer-based EPS severity ratings and in handwriting kinematic variables. The sample consisted of 21 SZ patients treated with risperidone, 6 unmedicated SZ subjects, and 47 healthy subjects. The unmedicated and risperidone-treated subjects exhibited comparable degrees of psychosis severity. Table 2 shows the descriptive statistics and ANOVA results for the three groups.

FIGS. 5 and 6 illustrate that, for example, average normalized jerk and vertical size in the sentence-writing task systematically change with daily dose of risperidone. Average normalized jerk (ANJ) increased with dose in the sentence condition (r=0.78; p<0.0001), while peak vertical velocity decreased with increasing dose for the sentence condition (r=−0.45; p<0.05). Each of these changes in kinematic variables is a marker of increased EPS. In contrast, there were no significant correlations (p>0.05) between the observer-based severity ratings and risperidone dose. These findings support the notion that objective handwriting measures are better suited to detect dose-related EPS than the conventional clinical severity ratings.

A multiple-regression analysis indicated that a 3-factor model consisting of vertical size, average normalized jerk, and velocity scaling together accounted for as much as 83% (r=0.91) of the variability in dose (F_{3,17}=27.27; p<0.00001).

### Conclusions

The invention out-performs conventional observer rating scales in discriminating important properties of medication type and dosage. These results demonstrate several new findings. First, that schizophrenia patients on atypical antipsychotics exhibit significant extrapyramidal motor impairment and that these impairments can be readily quantified using a naturalistic measure of handwriting involving a digitizing tablet and MovAlyzeR software program. Second, our findings show that some medications may be less effective than others in managing EPS. For example, in managing TD, our ANJ measure revealed that patients treated with quetiapine or olanzapine had significantly less abnormality than those treated with aripiprazole or risperidone. Moreover, we observed a dose-response relationship between risperidone and ANJ, a finding that has direct implications for managing antipsychotic-induced motor side effects. Third, while it has been recognized for some time that use of anticholinergic medications increases a patient's risk for developing TD, the present study confirms the deleterious effects on motor control using a simple handwriting task. Based on these findings, it is conceivable that handwriting quantification can be used in the clinical setting to identify patients at risk for developing troublesome EPS or for evaluating newer medications reaching the market. This study demonstrates that quantitative measures of handwriting could be useful in the clinical setting to identify patients at risk for developing troublesome EPS or for managing pharmacotherapy.

There were several highly significant medication group effects. These figures show that there are very interesting and useful findings collapsed across all 14 task conditions. FIGS. 1-3 of the document show that there are highly significant effects of antipsychotic type on average normalized jerk and peak velocity with those on the newest drug, called Abilify, doing worse than others. This is the first demonstration that this new drug may not be all that good as far as preventing EPS.

Risperidone-treated schizophrenia patients exhibit significant motor impairment and that these impairments can be readily quantified using a naturalistic measure of handwriting movements recorded by a digitizing tablet. Second, our results show that in a sentence-writing task, the severity of motor impairment as measured by three handwriting kinematic variables is significantly related to the average daily dose of risperidone. Specifically, decreased vertical stroke height, decreased peak vertical velocity and increased average normalized jerk account for 83% of the variability in daily risperidone dose. In contrast, conventional, observer-based EPS assessments appeared not able to detect differences between risperidone-medicated and unmedicated groups or a relationship with risperidone dose. These findings suggest that handwriting quantification could play a crucial role in the clinical setting when managing troublesome EPS and can offer valuable support for the clinician's decision to switch antipsychotics or alter dosage (c.f. Lambert, 2007).

### Example 2

### Effect of Risperidone Dosage

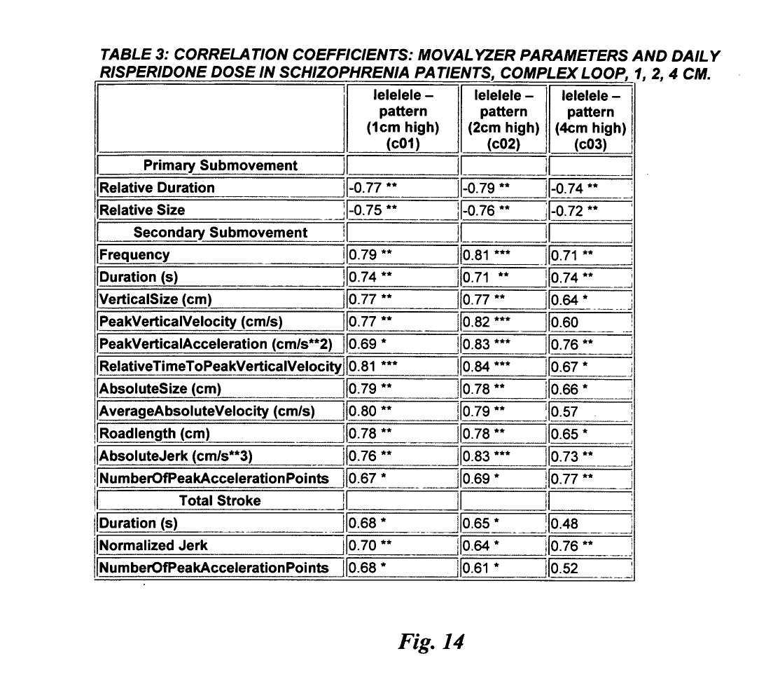

With reference to FIG. 14, Table 3 shows correlation coefficients (r) for relationship between MovAlyzeR parameters and daily dose of schizophrenia patients taking risperidone (mg/day) for the complex loop patters (cursive lleellee with vertical sizes 1, 2, or 4 cm). 21 subjects were included in the risperidone dose correlational analysis. Exclusion criteria included: concomitant use of anticholinergic medication, concomitant use of a conventional antipsychotics and daily dose greater than 7 mg. Inclusion criteria consisted of either gender, age between 18 and 65, DSM-IV diagnosis of schizophrenia or schizoaffective disorder (*p<0.05; **p<0.01; ***p<0.001).

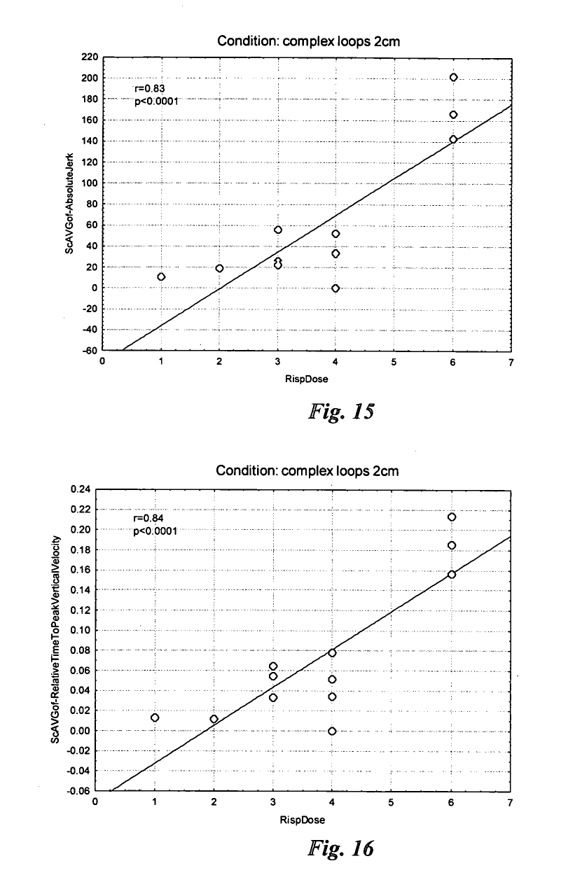

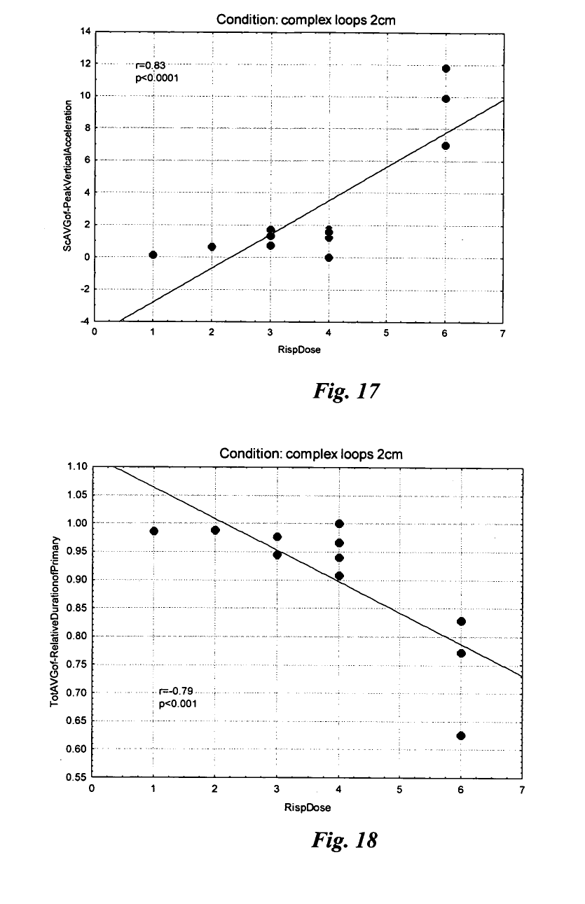

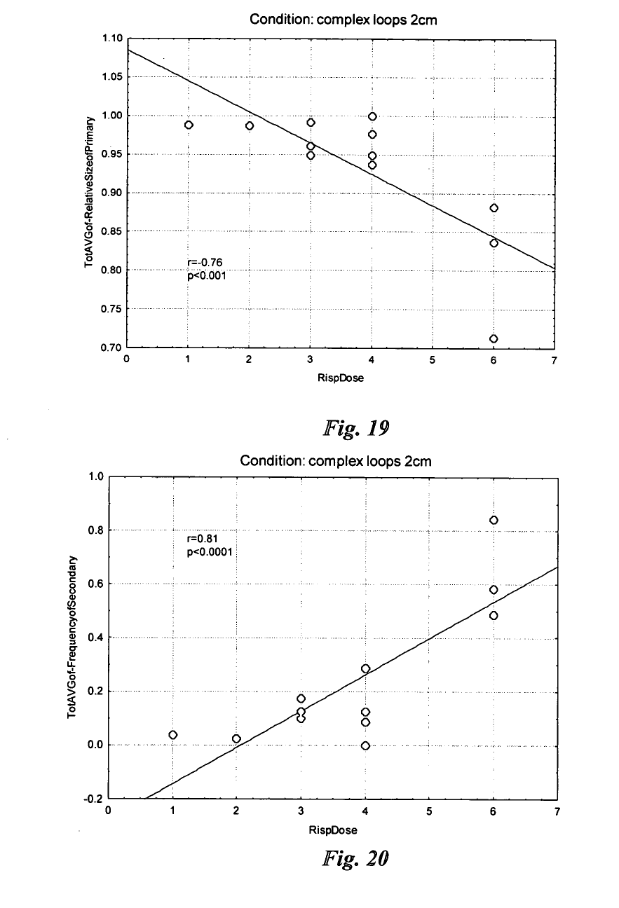

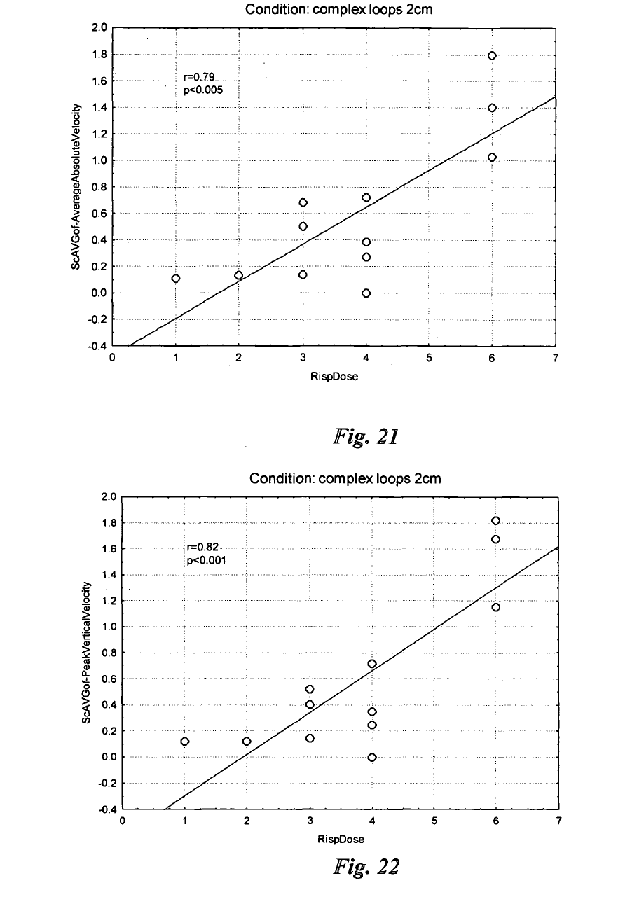

FIGS. 15-22 graph various extracted features of movement with respect to Risperidone dosage.

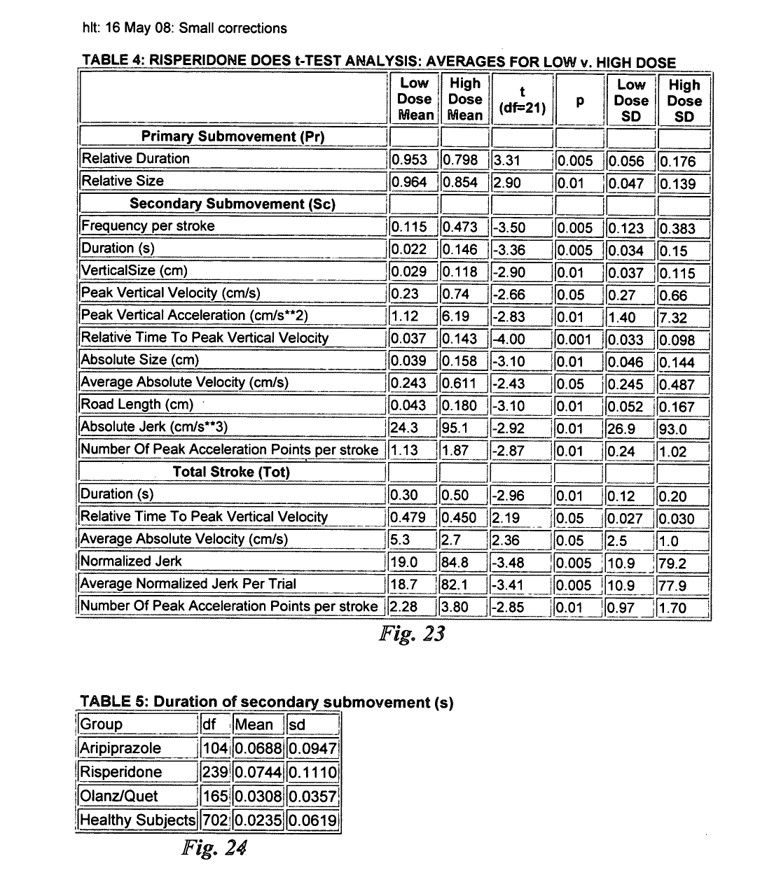

Considerable nonlinearity is found in the correlation charts. A clear division is apparent between low and higher dose. With reference to FIG. 23, Table 4, Student's t-tests were done to compare the averages for low dose versus those of the high dose. 23 subjects were included in this risperidone dose t-test analysis. Exclusion criteria included: concomitant use of anticholinergic medication and concomitant use of a conventional antipsychotics. There were 17 cases with daily dose 1-4 mg/day (low dose) and 6 cases with daily dose 5-10 mg/day. Inclusion criteria consisted of either gender, age between 18 and 65, DSM-IV diagnosis of schizophrenia or schizoaffective disorder.

### Effect of Medication Type

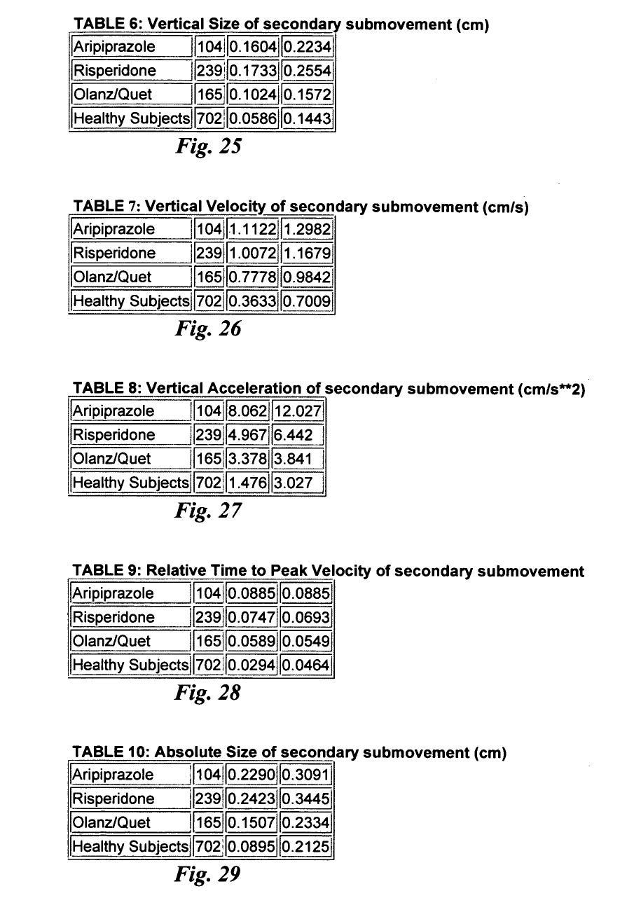

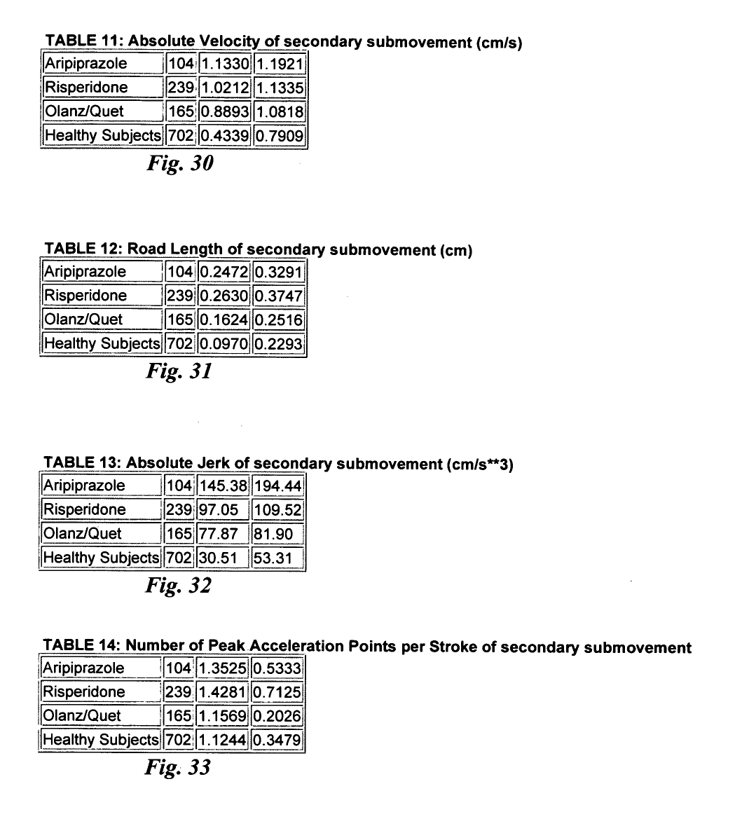

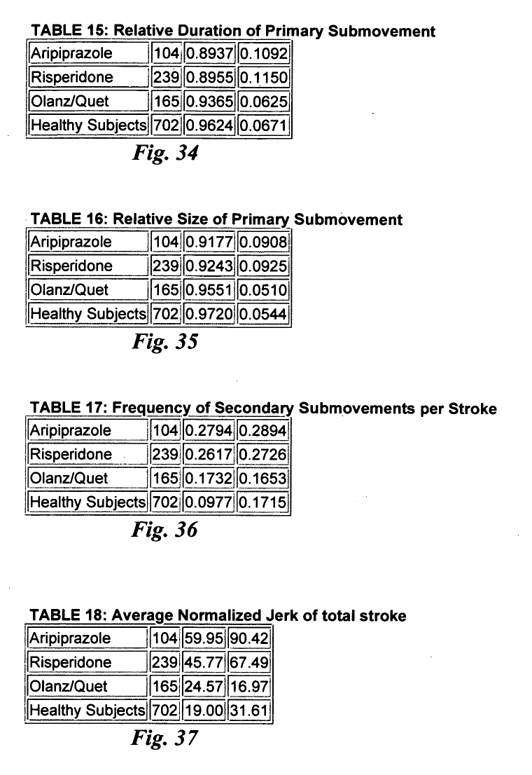

With reference to FIGS. 24-37, Tables 5-18, ANOVAs were run across patients grouped according to medication type (1=aripiprazole; 2=risperidone; 3=combined olanzapine/quetiapine) plus healthy controls. For the medication type analysis, we included 7 cases treated with aripiprazole; 16 with risperidone, and 11 with either quetiapine or olanzapine. Exclusion criteria included: concomitant use of anticholinergic medication and concomitant use of a conventional antipsychotics. Inclusion criteria consisted of either gender, age between 18 and 65, DSM-IV diagnosis of schizophrenia or schizoaffective disorder. 47 healthy subjects were included for comparison.

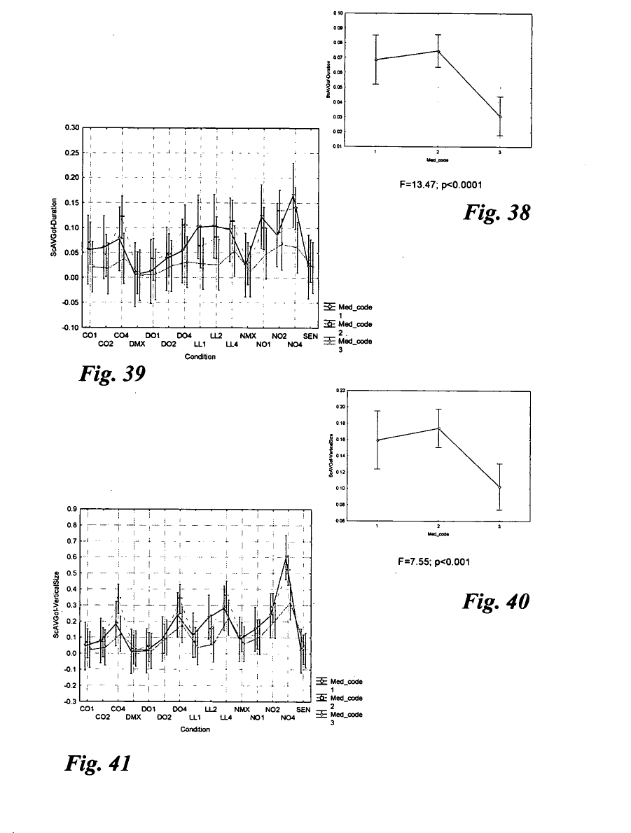

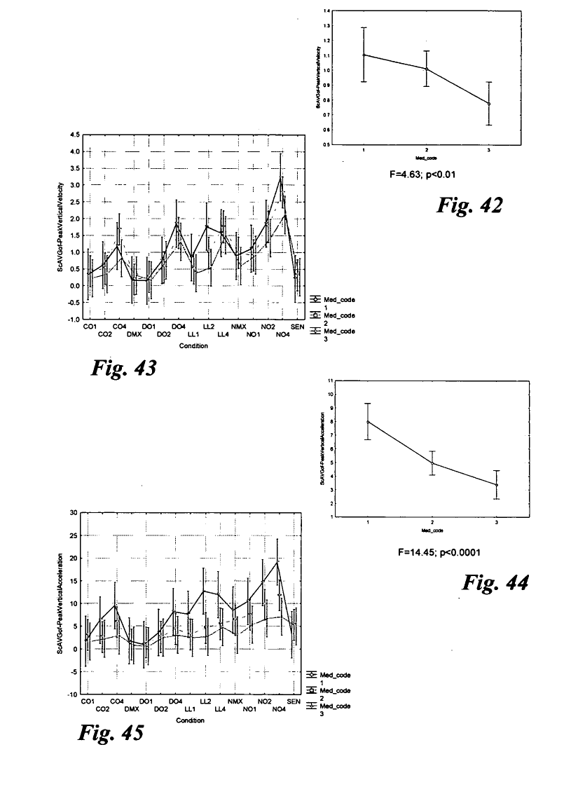

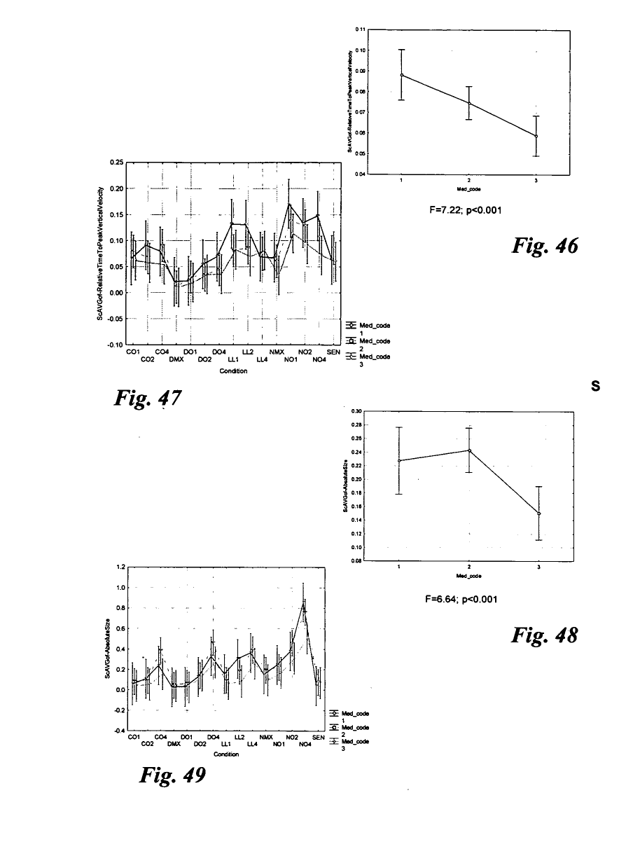

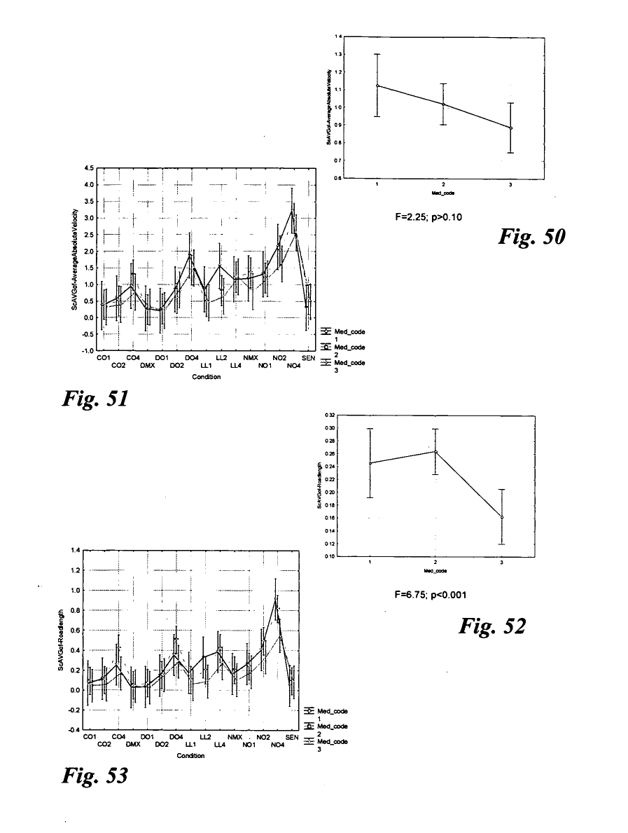

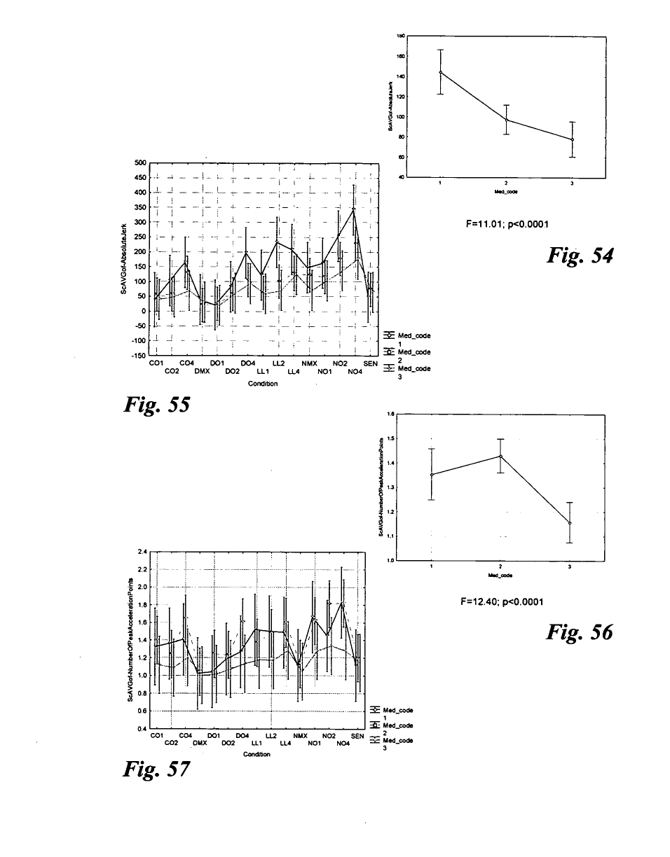

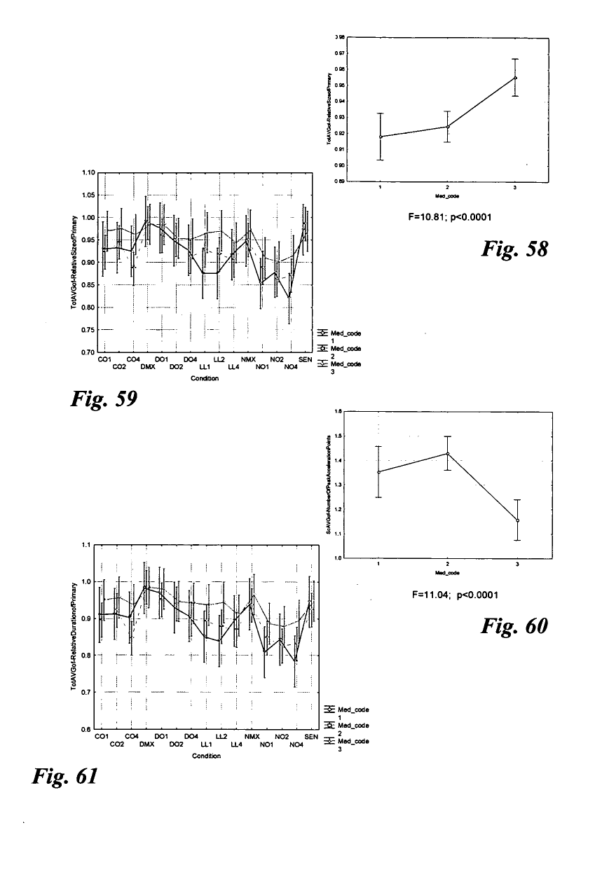

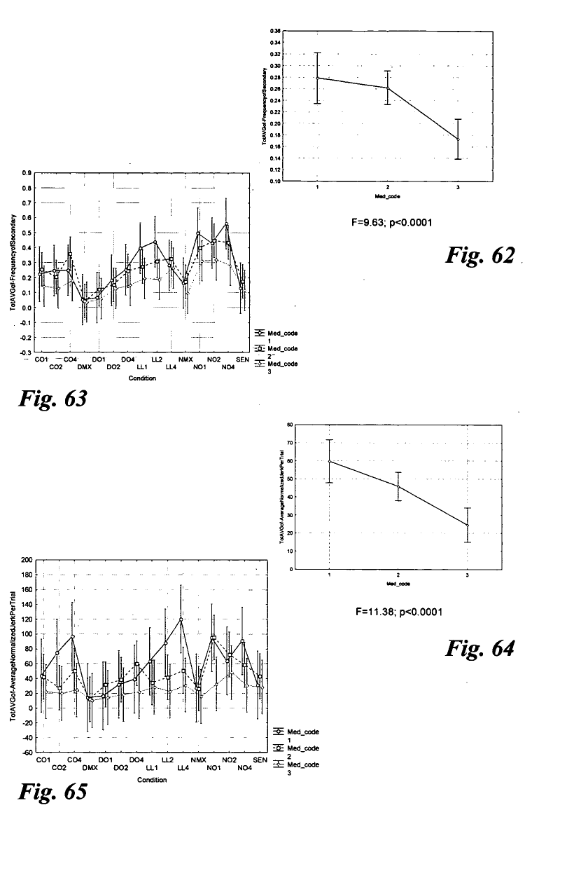

With reference to FIGS. 38-65, the graphs show the means and 95% confidence intervals of the 3 medication groups (upper right panels) and these values for each of the individual movement tasks: CO1, CO2 and CO4 are the complex cursive lleellee patterns at 1, 2, 4 cm vertical size, respectively. The exact magnitudes of the sizes are not relevant. We used 1, 2, 4 cm for convenience. We could have used two or more different sizes and call them qualitatively small versus large or small, mid, versus large. Only for the velocity scaling measure it is important that the sizes differ. The analysis measures the real size and the measure size, not the instructed size, will be used. The sizes are from guide lines fixed under an overhead sheet on the digitizer. DMX=Dominant-hand maximum-speed overlapping circles at 2 cm (8 circles), DO1, DO2 and DO4 are the dominant hand overlapping circles of 1, 2, or 4 cm vertical size, respectively. LL1, LL2 and LL4 are the cursive 11111111-patterns at 1, 2, 4 cm vertical size, respectively. NMX is the non-dominant hand maximum-speed overlapping circles at 2 cm. NO1, NO2 and NO4 are the nondominant hand overlapping circles at 1, 2, 4 cm vertical size. SEN is the sentence “Today is a nice day” at 2 cm vertical size.

### INDUSTRIAL APPLICABILITY

Our invention helps clinicians to identify patients who are particularly vulnerable to motor side effects caused by medication types or dosages that are not appropriate for them. The compelling reason to use our invention is that clinicians have many medications to choose from. Each type of medication has its problems in that it can cause a variety of disadvantages (motor side effects, metabolic side effects, high cost of certain types of medication). The physician needs information to base his decision on which medication to prescribe. For patients at risk for motor problems, he/she will better be able to avoid medication types or dosages that produce motor side effects. Small footprint, ease of use, and low cost of the required off-the-shelf hardware suit the present invention to deployment in primary care settings. Automation and computer-driven tests and instructions tend to reduce administrator bias and suit the present invention to delegating testing to less skilled practitioners. When used routinely, psychiatrists will easily be able to monitor or detect longitudinal changes in the patient's movements earlier than they could do presently with conventional observer-based rating scales. Besides schizophrenia patients, persons suffering from other nervous and mental diseases and other medication effects may benefit.

Other classes of mental and nerve diseases that can benefit of this invention are listed below. These patients often use psychotropic medications to control agitation, improve sleep, and reduce mania or anxiety:

Depression

Alzheimer's disease (with agitation)

Parkinson's disease

Bipolar disorder

Sleep disorders

Generalized Anxiety Disorder

Individuals treated with other classes of medications can benefit from this invention because these medications have one or more of the following pharmacodynamical activities: 1) Blocking dopamine: 2) Increasing availability of serotonin; 3) Blocking acetylcholine:

SSRI Antidepressants,

Antipsychotics

Mood stabilizers

Anticholinergics

Anxiolytics

Anticholinergic medications such as Benadryl or diphenhydramine

Antiemetic Medications such as metoclopramide (Reglan)

### Examples of Parkinson's Disease

We observed marked stroke duration changes during the medication cycle in Parkinson's disease patients. FIG. 67 graphs upstroke duration with respect to time during medication cycle in medicated Parkinson's disease patients. Before the Parkinson medication kicks in, stroke duration is large, within 1 hour stroke durations decrease dramatically and stay small until the medication is exhausted after a few hours towards the end of their medication cycle at which point stroke duration increases. This is the time the patient needs to take new medication (Poluha et al., 1998) (See Figure).

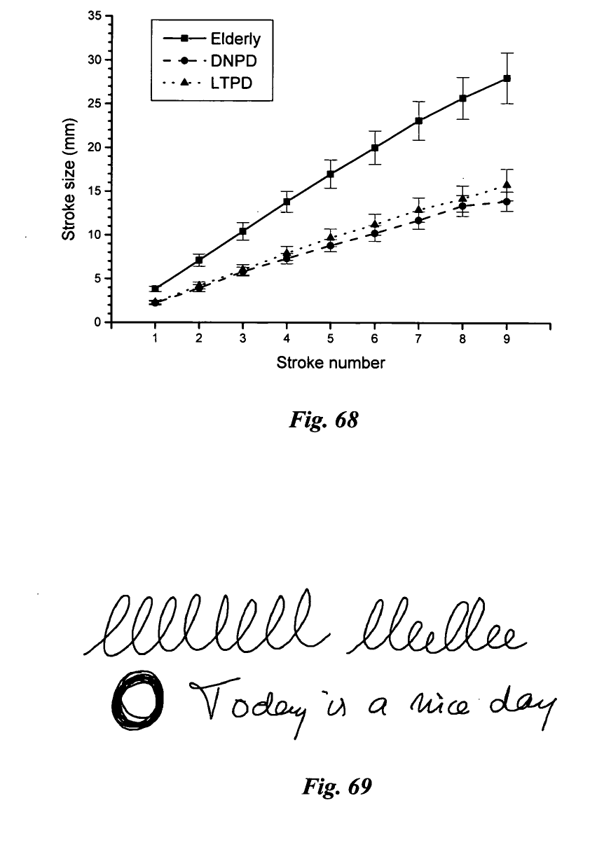

In another example on Parkinson's disease (Adler et al., 1997; 2003), we found that when patients were drawing an Archimedes Spiral, a common task used in neurologic evaluation, de Novo Parkinson patients (i.e., new patients who never received anti-Parkinson treatment) drew the Archimedes spiral much slower than the treated Parkinson patients (who on average should be more severely affected because they suffer already for a longer time from the progressive disease. FIG. 68 graphs stroke size with respect to stroke number in these three classes of patients.

### Problems Solved

The present invention enables a practitioner to:
1) Measure and immediately evaluate movement side effects due to medications in psychiatric and neurological patients
2) Help selecting appropriate drugs and dosages, especially neuroleptic medications
3) Reduce risk of patients developing permanent movement disorders from long-term use of inappropriate medication or dosage
4) Provide better and more cost-effective health care
5) Measure movement abnormalities along a linear continuum of severity
6) Exploit the superior sensitivity of instrumental observation to reveal subtle motor abnormalities below the threshold of detection by clinical evaluation
7) Potentially reveal subclinical motor phenomena
8) Obtain assessments which are more reliable than observer-based ratings scales because they do not depend on examiner experience and are not subject to examiner bias
9) Reduce medication noncompliance due to undesirable side effects. Increased adherence to medication regimen will reduce hospitalization and associated costs.
10) Use the present invention in psychiatric practices both for individual patient monitoring and for large multi-site clinical trials

Thus, in a method of assessing a motor sign in a human patient in accordance with the present invention a recording is established of the patient's performance of a motor task and a degree of the motor sign is inferred from the recording. This may be done by deriving a measure of dysfluency from the recording and inferring the degree from the measure of dysfluency. It may also be done by deriving a measure of velocity scaling from recordings of the patient drawing a plurality of figures of different sizes and inferring the degree from the measure of velocity scaling. The degree in some methods in accordance with the present invention is a degree of severity of a motor sign that is directly mediated by the central nervous system of the patient.

In some methods in accordance with the present invention, the degree of dysfluency may express a measure of average normalized jerk. This may be done, for example, by deriving a vertical movement component from the recording and deriving the measure of average normalized jerk from the vertical movement component.

Also in accordance with the present invention, a peak velocity increase and a vertical size increase may be derived from the recordings and a measure of velocity scaling may then be derived from the peak velocity increase and the vertical size increase.

Also in accordance with the present invention, a handwriting task includes a task such as drawing overlaying circles, drawing continuous loops, or drawing complex loops.

One preferred handwriting task, especially for deriving velocity scaling measures, is drawing loops having different vertical sizes varying between approximately one centimeter and approximately four centimeters.

Also in accordance with the present invention, method of detecting a drug side effect in a human patient includes ascertaining the patient's medication status with reference to a drug, establishing a recording of the patient's performance of a handwriting task, and inferring a degree of the drug side effect of the drug in the patient from the recording.

This may further include establishing a plurality of recordings of performances of the handwriting task by a plurality of patients in a population of patients, each such patient having known medication status with reference to the drug, and comparing the recording of the patient's performance with the plurality of recordings of performances by the population. The patient's medication status is then compared to that of other patients in the population. The motor task may include a handwriting task and the step of establishing the recording may include prompting the patient to write predetermined content with a writing instrument on a writing surface and acquiring data as the patient does so.

Also in accordance with the present invention, after the patient's performance of the motor task has been recorded, a measure may be derived from the recording, the measure being one of the following submovement parameters: frequency, Duration, VerticalSize, Peak Vertical Velocity, PeakVerticalAcceleration, Relative Time To Peak Vertical Velocity, Absolute Size, Average Absolute Velocity, Roadlength, Absolute Jerk, and Number Of Peak Acceleration Points. Then, the degree of the motor sign is inferred from the measure.

The degree being inferred may be a degree of severity of a motor sign such as bradykinesia or micrographia.

Exemplary apparatus in accordance with the present invention includes a handwriting recording device comprising a handwriting instrument and a handwriting surface. A computer is operatively coupled to the handwriting recording device and programmed to input data therefrom and to compute and report a measure of handwriting dysfluency from the data.

A handwriting template is usually added on top of the handwriting surface. The handwriting template comprising human-readable instructions for performing a handwriting task.

In an exemplary embodiment, the computer may be programmed to derive a measure of velocity scaling from the data and also to infer a degree of a motor sign in the patient from the data. The computer may be programmed to report a measure of average normalized jerk. The computer may be programmed to compute the measure of average normalized jerk based on a vertical movement component in the data.

### REFERENCE SIGNS LIST

testing procedure start condition 22
questionnaire 26
show-test-instructions 28
show-task-i-instructions 30
show-trial-j-instructions 32
recording starts 34
process trial 36
accept trial 38
all trials done 40
all tasks done 42
summarize 44
exit 46
start writing 48
input device 50
personal computer 52
raw device data 54
Time functions module 56
processed data 58
segmentation 60
segmentation points 62
Extraction 64
extracted features per stroke 66
Consistency checking 68
consistent extracted features per stroke 70
Summarize trials 72
consistent extracted summarized data 74
Analyze all data 76
analysis file 78
report 80
evaluation step 82
time function start 84
read 86
discontinuity 88
omit trailing pen lift 90
cyclic 92
transformation to frequency domain 94
power spectrum 96
unfiltered power spectrum 98
low-pass filter 100
copy of power spectrum 102
filtered power spectrum 104
reverse FFT 106
low-pass-filtered x and y with raw z data 108
velocity estimation 110
Velocity estimation 110
reverse FFT 112
output of low-pass-filtered velocities 114
absolute velocities 116
low-pass-filtered absolute velocities 118
acceleration estimation 120
reverse FFT 122
low-pass-filtered accelerations ax and ay 124
absolute accelerations 126
low-pass-filtered absolute accelerations 128
jerk estimation 130
reverse FFT 132
low-pass-filtered jerk jx and jy 134
absolute jerks 136
low-pass-filtered absolute jerks 138
finish 140
segmentation start 142
read 144
find 146
step search forward 148
step search backwards 150
step search backwards from end 152
search forward 154
step search forward 156
reached last 158
interpolate 160
remove 162
step submovement 164
step scale 166
finish 168
extraction start 170
read 172
duration 174
vertical size 176
peak velocity 178
normalized jerk 180
output 182
finish 184

### Citation List

Patent Literature
U.S. Pat. No. 6,632,174
U.S. Pat. No. 6,546,134
U.S. Pat. No. 6,454,706
U.S. Pat. No. 5,772,611
U.S. Pat. No. 5,730,602
Non Patent Literature
Adler, C. H., Teulings, H. L., & Stelmach, G. E. (1997). Handwriting abnormalities in Parkinson's disease and response to treatments. Movement Disorders, 12, Supplement 1, 47.
Adler, C. H., Van Gemmert, A. W. A., Teulings, H. L., Stelmach, G. E. (2003). A quantitative analysis of the production of the Archimedes spiral in Parkinson's disease patients and controls. H. L. Teulings, Van Gemmert, A. W. A. (Eds.), Proceedings of the 11th Conference of the International Graphonomics Society (IGS2003), 2-3 Nov. 2003, Scottsdale, Ariz., USA. ISBN 0-9746365-0-9. (p. 119-122).
Caligiuri M P, Jeste D V, Lacro J P (2000) Antipsychotic-induced movement disorders in the elderly: epidemiology and treatment recommendations. Drugs and Aging 17: 363-384.
Caligiuri M P, Teulings, H L, Filoteo, J V, Song, D, Lohr, J B (2006). Quantitative measurement of handwriting in the assessment of drug-induced parkinsonism. Hum Mov Sci. October; 25 (4-5):5 10-22.
Caligiuri et al., (submitted) Handwriting Movement Analyses for Monitoring Drug-Induced Motor Side Effects in Schizophrenia Patients Treated with Risperidone. Human Movement Science.
Caligiuri M P, Teulings H L, Dean C, Niculescu A, Lohr J B (2007) Handwriting quantification for monitoring drug-induced EPS in schizophrenia. Paper presented to Society for Biol Psychiatry, May 18, 2007
Caligiuri, M. P., Teulings, H. L., Dean, C. E., Niculescu, A. B., Lohr, J. B. (submitted). Handwriting Movement Analyses for Monitoring Drug-Induced Motor Side Effects in Schizophrenia Patients Treated with Risperidone. Submitted for publication in the special IGS issue of Human Movement Sciences.
Caligiuri, M. P., Teulings, H. L., Dean, C. E., Niculescu, A. B., Lohr, J. B. (submitted). Handwriting Movement Kinematics in Psychosis Patients With Drug-induced Parkinsonism. Submitted for publication in Schizophrenia Bulletin.
Caligiuri, M. P. & Teulings, H. L. (2007). Movement analysis system to monitor fine motor abnormalities in persons with neuropsychiatric diseases. Provisional U.S. Patent 60/938,409. 16 May 2007.
Caligiuri, M. P., Teulings, H. L., Dean, C. E., Niculescu, A. B., Lohr, J. B. (2007). Handwriting Movement Analyses For Monitoring Drug-Induced Motor Side Effects In Schizophrenia In Phillips, J. G., Rogers, D., Ogeil, R. P. (Eds.), Proceedings of the 13th Conference of the International Graphonomics Society (1GS2007). 11-14 Nov. 2007, Melbourne, VIC, Australia. ISBN 978-0-7326-4003-3. pp. 75-78.
Caligiuri, M. P., Teulings, H. L., Dean, C. E., Niculescu, A. B., Lolir, J. B. (2007). (Poster). Handwriting Movement Analysis to Monitor Drug-Induced Movement Side Effects in Schizophrenia Patients. AzBioExpo2007, 20 Jun. 2007.
Caligiuri, M. P., Teulings, H. L., Dean, C. E., Niculescu, A. B., Lohr, J. B. (2007). (Poster). Handwriting Quantification for Monitoring Drug-Induced BPS in Schizophrenia. 62nd Annual Conference of the Society of Biological Psychiatry, San Diego, Calif., May 17-19, 2007. Published in: Biological Psychiatry 2007; 61:15-2665 p. 169S
Caligiuri, M. P., Teulings, H. L., Dean, C. E., Niculescu, A. B., Lohr, J. B. (2007). (Poster). Handwriting Quantification For Monitoring Drug-Induced Extrapyramidal Side Effects In Patients With Psychosis. 160th Annual Meeting of the American Psychiatric Association, San Diego, Calif., May 19-24, 2007. p. 188.
Carlton, L. G. (1980). Movement control characteristics of aiming responses. Ergonomics, 23, 1019-1032.
Crossman, E. R. F. W., & Goodeve, P. J. (1963/1983). Feedback control of hand-movement and Fitts' law. Quarterly Journal of Experimental Psychology, 35A, 251-278 (original work presented at Experimental Psychology Society, Oxford, England, 1963).
De Jong, Hulstijn, Kosterman, Smits-Engelsman (1996) in: M. L. Simner, C. G. Leedham, A. J. W. M. Thomassen (Eds). Handwriting and Drawing Research: Basic and Applied Issues. Amsterdam: IOS.
Gerken A, Wetzel H, Benkert O (1991) Extrapyramidal symptoms and their relationship to clinical efficacy under perphenazine treatment. A controlled prospective handwriting-test study in 22 acutely ill schizophrenic patients. Pharmacopsychiatry 24: 132-137.
Gierhart, H. S., & Teulings, H. L. (1995). Teaching handwriting in the computer age. Proceedings of the 7th International Graphonomics Society Conference (pp. 58-59), London, Ontario, Canada, August 1995, ISBN 0-921121-14-8.
Haase H J (1978) The purely neuroleptic effects and its relation to the “neuroleptic threshold”. Acta Psychiatr Belg 78: 19-36.
Janno S, Holi M M, Tuisku K, Wahlbeck K. (2005). Validity of Simpson-Angus Scale (SAS) in a naturalistic schizophrenia population. BMC Neurol. 2005 Mar. 17; 5(1):5.
Janno S, Holi M M, Tuisku K, Wahlbeck K. (2005). Actometry and Barnes Akathisia Rating Scale in neuroleptic-induced akathisia. Eur Neuropsychopharmacol. 2005 January; 15(1):39-41.
Mai et Marquardt, Experimental Brain Research, 1999; 128: 224-228
Kuenstler U, Juhhold U, Knapp W H, Gertz H H (1999) Positive correlation between reduction in handwriting area and D2 dopamine receptor occupancy during treatment with neuroleptic drugs. Psychiat Research 90: 31-39.
Lambert T J (2007) Switching antipsychotic therapy: what to expect and clinical strategies for improving therapeutic outcomes. J C/in Psychiatty. 68 Suppl 6:10-13.
Meyer, D. E., Smith, J. E. K., Kornblum, S., Abrams, R. A., & Wright, C. E. (1990). Speed-accuracy tradeoffs in rapid aimed movements: Toward a theory of rapid voluntary action. In M. Jeannerod (Ed.), Attention and Performance XIV (pp. 173-226). Hillsdale, N.J.: Lawrence Erlbaum Associate.
Meyer, D. E., Abrams, R. A., Kornblum, S., Wright, C. E., & Smith, J. E. K. (1988). Optimality in human motor performance: Ideal control of rapid aimed movements. Psychological Review, 95, 340-370.
Pfann K D, Buchman A S, Comella C L, Corcos D M (2001) Control of movement distance in Parkinson's disease. MovDis 16: 1048-1065.
Teulings H L, Maarse F J (1984) Digital recording and processing of handwriting movements. Hum Move Science, 3, 193-217.
Teulings, H. L., Czerepacha, H. T., Romero, D. H., Subramanian, R. (2005). Handwriting teaching tool for children. Proceedings of the 12th Biennial Conference of the International Graphonomics Society (pp 95-99), 26-29 Jun. 2005, Salerno, Italy. Civitella, Italy: Editrice Zona.
Teulings, H. L., Contreras-Vidal, J. L., Stelmach, G. E., and Adler, C. H. (1997). Coordination of fingers, wrist, and in Parkinsonian handwriting. Experimental Neurology, 146, 159-170.
Walker, N., Meyer, D. E., Smelcer, J. B. (1993). Spatial and temporal characteristics of rapid cursor-positioning movements with electromechanical mice in human-computer interaction. Human Factors, 35, 431-458.
Walker, N., Philbin, D. A., Fisk, A. D. (1997). Age related differences in movement control: adjusting submovement structure to optimize performance. Journal of Gerontology; Psychological Science, 52B, 40-52.
Poluha, P. C., Teulings, H. L., Brookshire, R. H. (1998). Handwriting and speech changes across the levodopa cycle in Parkinson's disease, Acta Psychologica, 100, 71-84.

1. A method of assessing a degree of severity of a motor sign directly mediated by a pharmacological agent in a human patient, comprising the steps of:
a. establishing a recording of the patient's performance of a motor task;
b. deriving from said recording a measure selected from the group including a measure of dysfluency and a measure of bradykinesia; and
c. inferring a degree of the motor sign from said measure.

2. (canceled)

3. The method of claim 1, wherein said motor task is a handwriting task comprising writing a handwriting-like pattern selected from the group containing overlaying circles, continuous loops, complex loops, and a sentence.

4. A method of assessing a motor si n in a human patient, comprising the steps of:
a. establishing a recording of the patient's performance of a motor task;
b. deriving from said recording a vertical or horizontal movement component in the writing plane; and
c. inferring a degree of the motor sign from said measure.

5. A method of assessing a motor sign in a human patient, comprising the steps of:
a. establishing recording of the patient's performance of a motor task;
b. deriving from said recording a measure of dysfluency or a measure of bradykinesia; and
c. inferring a degree of the motor sign from said measure,
wherein said step of establishing said recording further comprises prompting the patient to write predetermined content with a writing instrument on a writing surface and acquiring recorded data as the patient does so.

6. A method of assessing a motor sign in a human patient, comprising the steps of:
a. establishing a recording of the patient's performance of a motor task, said task comprising a handwriting task of drawing a plurality of figures of different sizes;
b. deriving a peak velocity increase and a vertical size increase in the writing plane from said recording; and
c. inferring a degree of the motor sign from said peak velocity increase and said vertical size increase.

7. (canceled)

8. A method of assessing a motor sign in a human patient, comprising the steps of
a. establishing a recording the patient's performance of motor task, said task comprising hand-writing a plurality of figures of different sizes;
b. deriving a measure of velocity scaling from said recording; and
c. inferring a degree of the motor sign from said measure of velocity scaling,
wherein the figures have loops with vertical sizes between approximately one centimeter and approximately four centimeters.

9. The method of claim 8, wherein said plurality of figures comprise at least one figure selected from the group containing overlaying circles, continuous loops, complex loops, and sentences.

10. A method of assessing a motor sign in a human patient, comprising the steps of:
a. establishing a recording of the patient's performance of a motor task, said task comprising a handwriting task of drawing a plurality of figures of different sizes;
b. deriving a measure of velocity scaling from said recording; and
c. inferring a degree of the motor sign from said measure of velocity scaling,
wherein said step of establishing said recording further comprises prompting the patient to write predetermined content with a writing instrument on a writing surface and recording data as the patient does so.

11. (canceled)

12. A method of detecting a drug side effect in a human patient, comprising the steps of:
a. ascertaining the patient's medication status with reference to a drug;
b. establishing a recording of the patient's performance of a handwriting task;
c. deriving from said recording at least one measure selected from the group containing a measure of dysfluency and a measure of bradykinesia;
d. establishing a plurality of recordings of performances of said handwriting task by a plurality of patients in a population of patients, each such patient having known medication status with reference to said drug;
e. deriving from said plurality of recordings at least one measure selected from the group containing a measure of dysfluency and a measure of bradykinesia;
f. making a first comparison of said at least one measure derived from said recording of the patient's performance with said at least one measure derived from said recordings of said performances by said plurality of patients in said population;
g. making a second comparison of the patient's medication status to said medication status of said plurality of patients; and
h. inferring a degree of the drug side effect of said drug in the patient from said first comparison and said second comparison.

13. The method of claim 12, wherein said handwriting task comprises writing a plurality of figures of different sizes and said measure is a measure of velocity scaling.

14. The method of claim 13, wherein said measure is computed with reference to size increase in different ones of figures drawn in said task of drawing a plurality of figures of different sizes.

15. Apparatus for assessing a motor sign in a human patient, comprising:
a. a handwriting recording device comprising a handwriting instrument and a handwriting surface; and
b. a computer operatively coupled to said handwriting recording device and programmed to input data therefrom and to compute from said data and report a measure of a motor sign indicating dysfluency or bradykinesia.

16. Apparatus as set forth in claim 15, wherein said computer is programmed to identify one or more submovements as represented by said recording and to derive from said identified submovements at least one measure selected from the group of measures including measures comprising Relative Duration and Relative Size of the Primary Submovement, Secondary Submovement Frequency, Duration, Vertical Size, Peak Vertical Velocity, Peak Vertical Acceleration, Relative Time To Peak Vertical Velocity, Absolute Size, Average Absolute Velocity, Roadlength, Absolute Jerk, and Number Of Peak Acceleration Points.

17. A method of assessing a motor sign in a human patient, the method comprising the steps of:
a. with a data processing apparatus, interacting with the patient to communicate to the patient a task and prompting the patient to perform said task;
b. with the data processing apparatus interacting with the patient, establishing a recording over a first time period having an end defined as cessation of movement or as the expiration of a predetermined time period;
c. with the data processing apparatus, testing said recording for a condition that said recorded performance is consistent with said communicated task;
d. with the data processing apparatus, unless said condition is true, rejecting said recording and again prompting the patient to perform said communicated task;
e. accepting said recording and appending said recording to a collection of data;
f. repeating the aforementioned steps of communicating, prompting, establishing, testing and accepting until said recording has been established and accepted a predetermined number of times;
g. deriving from said collection of recorded data a measure selected from the group containing a measure of dysfluency and a measure of bradykinesia; and
h. inferring a degree of the motor sign from said measure.

18. The method of claim 17, wherein said degree of the motor sign is a degree of severity that is directly mediated by a pharmacological agent.

19. The method of claim 17, wherein said motor task is a handwriting task comprising writing a handwriting-like pattern selected from the group containing overlaying circles, drawing continuous loops, complex loops, and a sentence.

20. The method of claim 17, further comprising the steps of identifying one or more submovements as represented by said recording; and deriving, from said recording and with reference to said one or more submovements, at least one measure selected from the group of submovement parameters consisting of Relative Duration and Relative Size of the Primary Submovement, Secondary Submovement Frequency, Duration, Vertical Size, Peak Vertical Velocity, Peak Vertical Acceleration, Relative Time To Peak Vertical Velocity, Absolute Size, Average Absolute Velocity, Roadlength, Absolute Jerk, Normalized Jerk, Average Normalized Jerk, and Number Of Peak Acceleration Points; and inferring a degree of the motor sign from said measure.

21. A method of monitoring a patient's compliance with a medication regimen, comprising the steps of:
a. establishing a recording of the patient's performance of a motor task, said task comprising hand writing a plurality of figures of different sizes;
b. deriving from said recording a plurality of peak values selected from the group containing velocity values and duration values, each with respect to one or more figures of different sizes;
c. computing a quotient by dividing said peak values by corresponding ones of different sizes;
d. comparing said quotient with one or more values of said quotient obtained from a reference set, said reference set being selected from the group containing a set of quotient values obtained from a population of patients and a set of quotient values obtained from the same patient at different times, said reference set being associated with data pertaining to compliance with a medication regimen; and
e. inferring the patient's compliance status from said comparison.

22. A method of monitoring a patient's compliance with a medication regimen, comprising the steps of:
a. establishing a recording of the patient's performance of a motor task, said task comprising hand writing a plurality of figures of different sizes;
b. deriving from said recording a plurality of relative duration values each with respect to one or more figures of different sizes;
c. computing a quotient by dividing said relative duration values by corresponding ones of different sizes;
d. comparing said quotient with one or more values of said quotient obtained from a reference set, said reference set being selected from the group containing a set of quotient values obtained from a population of patients and a set of quotient values obtained from the same patient at different times, said reference set being associated with data pertaining to compliance with a medication regimen; and
e. inferring the patient's compliance status from said comparison.

23. The method of claim 1, wherein said measure of dysfluency is computed as the number of acceleration peaks per stroke in said recording and said measure is used to infer a movement side effect.

24. The method of claim 12, wherein said measure of dysfluency is computed as the number of acceleration peaks per stroke in said recording and said measure is used to infer a movement side effect.

25. The method of claim 1, wherein step b further comprises
identifying in said recording a stroke corresponding to a movement performed by the patient;
identifying with reference to said stroke a stroke duration, a first velocity peak, and an end;
identifying between said first velocity peak and said end a first negative-to-positive zero crossing of acceleration;
determining the time elapsed between said zero crossing and said end; and
deriving said measure from a quotient of said time elapsed divided by the stroke duration.

26. The method of claim 1, wherein step b further comprises
identifying in said recording a stroke corresponding to a movement performed by the patient;
determining with reference to said stroke a stroke duration and a stroke size;
computing with reference to said stroke an integral, across the entirety of said stroke, of the third time derivative of said stroke;
obtaining the product of said integral multiplied by the fifth power of said duration;
obtaining the quotient of said product divided by the second power of said stroke size; and
deriving said measure from said quotient, thereby obtaining the measure known as normalized jerk.

27. The method of claim 12, wherein step c comprises determining with reference to said recording a duration of a task performed by the patient; step e comprises determining with reference to said plurality of recordings by said plurality of patients a plurality of durations; and step f comprises deriving a measure of bradykinesia by comparing said duration with said plurality of durations.

28. The method of claim 12, wherein step c comprises determining with reference to said recording a stroke size of a task performed by the patient; step e comprises determining with reference to said plurality of recordings by said plurality of patients a plurality of stroke sizes; step f comprises said stroke duration with said plurality of stroke sizes in said population.

29. A method of assessing a motor sign in a human patient, comprising the steps of:
a. establishing a recording of the patient's performance of a motor task entailing drawing a plurality of figures of different sizes;
b. deriving a peak velocity increase and a vertical size increase from said recording; and
c. inferring a degree of the motor sign from said peak velocity increase and said vertical size increase.

30. A method of assessing a motor sign in a human patient, comprising the steps of:
a. establishing a recording of the patient's performance of a motor task;
b. deriving a measure of dysfluency from said recording; and
c. inferring a degree of the motor sign from said measure of dysfluency, wherein said measure of dysfluency is derived with reference to a computation of average normalized jerk.

31. The method of claim 30, wherein step b further comprises deriving a movement component from said recording and computing said measure of average normalized jerk from a horizontal movement component, a vertical movement component, and a resultant movement component.

32. (canceled)

33. A method of assessing a motor sign in a human patient, comprising the steps of:
a. establishing a recording of the patient's performance of a motor task entailing drawing plurality of figures of different sizes;
b. deriving a measure of velocity scaling from said recording; and
c. inferring a degree of the motor sign from said measure of velocity scaling,
wherein step a further comprises drawing loops having vertical sizes between approximately one centimeter and approximately four centimeters.
